(19) United States
(12) Patent Application Publication
(10) Pub. No.: US 2024/0245525 A1
(43) Pub. Date: Jul. 25, 2024

(54) DEVICES AND METHODS FOR VERTEBRAL BONE REALIGNMENT

(71) Applicant: Samy Abdou, San Diego, CA (US)

(72) Inventor: Samy Abdou, San Diego, CA (US)

(21) Appl. No.: 18/243,397

(22) Filed: Sep. 7, 2023

Related U.S. Application Data
(63) Continuation of application No. 17/683,167, filed on Feb. 28, 2022, now Pat. No. 11,752,008.
Application No. 17/683,167 is a continuation of application No. 17/222,896, filed on Apr. 5, 2021, now Pat. No. 11,259,935.
Application No. 17/222,896 is a continuation of application No. 16/780,815, filed on Feb. 3, 2020, now Pat. No. 10,973,648.
Application No. 16/780,815 is a continuation-in-part of application No. 15/793,895, filed on Oct. 25, 2017, now Pat. No. 10,744,000.
(60) Provisional application No. 62/496,721, filed on Oct. 25, 2016.

(51) Int. Cl. A61F 2/44; A61F 2/30

(52) U.S. Cl. CPC A61F 2/4425; A61F 2/4455; A61F 2002/30538; A61F 2002/3055; A61F 2002/30624; A61F 2002/443; A61F 2002/444

(57) ABSTRACT

Methods and apparatus for providing correction of one or more maladies or conditions of the spinal column of a living being. In one embodiment, the apparatus includes an implantable device configured to be selectively adjustable in one or more portions thereof so as to permit correction of asymmetries or irregularities of the spinal column via insertion into one or more affected intervertebral disc spaces. In one variant, the implantable device includes upper and lower host elements which are hinged or can pivot relative to one another, and an insertable distraction mechanism which is adjustable to enable one side or the other of the implantable device to alter height. In another variant, both sides of the implantable device can be adjusted for height via the host elements and one or more pivots or hinges. In one implementation, the distraction mechanism is adjustable from multiple approaches into the disc space.

### PRIORITY

This application is a continuation and claims the benefit of priority to co-owned and co-pending U.S. patent application Ser. No. 17/683,167 entitled “DEVICES AND METHODS FOR VERTEBRAL BONE REALIGNMENT” and filed Feb. 28, 2022 and issuing as U.S. Pat. No. 11,752,008 on Sep. 12, 2023, which is a continuation and claims the benefit of priority to co-owned U.S. patent application Ser. No. 17/222,896 of the same title filed Apr. 5, 2021 and issued as U.S. Pat. No. 11,259,935 on Mar. 1, 2022, which is a continuation and claims the benefit of priority to co-owned U.S. patent application Ser. No. 16/780,815 of the same title filed Feb. 3, 2020, and issued as U.S. Pat. No. 10,973,648 on Apr. 13, 2021, which is a continuation-in-part of and claims the benefit of priority to co-owned U.S. patent application Ser. No. 15/793,895 filed on Oct. 25, 2017 of the same title, issued as U.S. Pat. No. 10,744,000 on Aug. 18, 2020, which claims the benefit of priority to co-owned U.S. Provisional Patent Application Ser. No. 62/496,721 entitled “DEVICES AND METHODS FOR VERTEBRAL BONE REALIGNMENT”, filed Oct. 25, 2016, each of the foregoing of incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND

### 1. Field of the Disclosure

This disclosure relates generally to medical devices, and in one exemplary aspect to bone fixation systems, components thereof, and methods of implant placement, which can be used to, inter alia, adjust, align and maintain the spatial relationship(s) of adjacent bones or bony fragments during and/or after surgical reconstruction of skeletal segments. While illustrated for use in the vertebral column, it is understood that the disclosed implants and methods may be used in any application skeletal segment.

### 2. Description of Related Technology

Whether from congenital malformation, degenerative disease, traumatic disruption, infection or neoplastic invasion, alteration in the anatomical alignment between the spinal vertebrae can cause significant pain, deformity, neurological decline and disability. Spinal disease is a major health problem in the industrialized world and the surgical treatment of spinal pathology is an evolving discipline. The traditional surgical treatment of abnormal vertebral motion and/or formation is the complete immobilization and bony fusion of the involved spinal segment and an extensive array of surgical techniques and implantable devices have been formulated to accomplish the treatment objective.

Regardless of the specific objectives of surgery, many surgeons employ implantable devices that maintain the desired spatial relationship(s) between adjacent vertebral bodies. However, conventional implantable devices are limited in that they are, inter alia, primarily “one size fits all,” including standardized configurations and sizes which are non-adjustable and/or not particularly adapted for certain applications. Thus, such conventional implants may be insufficient for treatment of patients with unusual or complex spinal curvatures and maladies, which may occur in conditions such as e.g., coronal plane deformity (such as scoliosis), sagittal plane deformity (such as alternation in segmental kyphosis or lordosis), axial translation, spondylolisthesis, etc.

Hence there is a salient need for alternative methods and devices for the alteration and/or correction of spinal curvature, which, inter alia, enable variable and/or adjustable configurations for implantable devices, such as to realign adjacent vertebrae according to the spinal structure or curvature of a specific subject.

### SUMMARY

Improved devices, systems, and methods to alter vertebral alignment are described herein.

In one aspect, an implantable device is disclosed. In one embodiment, the device comprises a distraction mechanism, and a first member or a host member configured to at least partly retain the distraction mechanism.

In one variant, the host member comprises a set of substantially planar elements configured to articulate relative to one another around at least one axis, so as to permit insertion and/or removal of the distraction mechanism. The distraction mechanism can cause the implantable device to change a height of one side so as to allow for intervertebral correction of e.g., scoliosis.

In another variant, the host member is configured to enable adjustment of height of both sides of the implant.

In another aspect, a method of inserting an implantable device within an intervertebral space is disclosed. In one embodiment, the method includes inserting an assembled implant device in the disc space, and adjusting a height of at least a portion thereof so as to compensate for asymmetries in the disc space caused by, e.g., scoliosis.

In another aspect, a method of treating a spinal misalignment is disclosed. In one embodiment, the method includes utilizing an implant assembly to alter the spinal alignment of a target functional spinal unit in the coronal plane in order to treat coronal plane deformities such as, e.g., scoliosis.

In another embodiment, the method includes utilizing an implant assembly to alter the spinal alignment of a target functional spinal unit in the sagittal plane in order to treat sagittal plane deformities such as, e.g., abnormal lordosis and/or kyphosis.

In yet another embodiment, the method comprises utilizing an implant assembly to alter the spinal alignment of the target functional spinal unit in the axial plane in order to, for example, treat translational deformities such as e.g., anterior/posterior or lateral spondylolisthesis.

In another aspect, a distraction mechanism for use within an implantable device is disclosed. In one embodiment, the distraction mechanism includes a piston which utilizes a working fluid to drive the piston (and hence a top portion of the mechanism) into compressive contact with an inferior surface of a superior vertebral segment. In another embodiment, the mechanism uses a mechanical (non-fluidic) arrangement for the compression (e.g., worm drive, gear mechanism, etc.).

In a further aspect, a system for correction of spinal conditions is disclosed. In one embodiment, the system includes: (i) a host housing member, (ii) a distraction mechanism, and (iii) a tool for adjusting the distracting mechanism after implantation.

### BRIEF DESCRIPTION OF THE DRAWINGS

FIGS. 1A-1C show diagrammatic representations of a spinal vertebral bone in multiple views.

FIGS. 2A and 2B illustrate a functional spinal unit (FSU), which includes two adjacent vertebrae and the intervertebral disc between them.

FIG. 3 shows a schematic representation of the posterior aspect of a patient who is positioned in a lateral decubitus orientation.

FIG. 4 illustrates a cross sectional view of the torso at the level of a targeted disc space in the lumbar spine.

FIG. 5 illustrates a cross sectional view of a targeted disc space in the lumbar spine, illustrating various approaches thereto.

FIGS. 6A and 6B illustrate one embodiment of an implantable device according to the present disclosure.

FIG. 7A shows top and side plan views of the device of FIGS. 6A and 6B, with distraction mechanism inserted.

FIG. 7B shows a side plan view of the device of FIGS. 6A and 6B, in a closed position.

FIG. 7C shows a side plan view of the device of FIGS. 6A and 6B, in an open position, with distraction mechanism installed.

FIG. 8A shows a top perspective view of the device of FIGS. 6A and 6B.

FIG. 8B shows a side elevation view of the device of FIG. 8A.

FIG. 8C shows a front elevation view of the device of FIG. 8A.

FIG. 9A shows a top perspective view of one embodiment of the distraction mechanism of FIGS. 6A and 6B.

FIG. 9B shows a top view of the distraction mechanism of FIG. 9A.

FIG. 9C shows a front view of the distraction mechanism of FIG. 9A.

FIG. 9D shows a side view of the distraction mechanism of FIG. 9A.

FIGS. 10A, 10B, 10C, and 10D show top, top perspective, and side views of various constituent members of the distraction mechanism of FIG. 9.

FIG. 11A shows a top perspective view of the exemplary distraction mechanism of FIG. 9.

FIG. 11B shows a front cross-sectional view (taken along line B-B) of the exemplary distraction mechanism of FIG. 9, shown positioned within a host member of the implant device.

FIG. 11C shows a side plan view of the device of FIGS. 6A and 6B.

FIG. 11D shows a front cross-sectional view (taken along line B-B) of the device of FIGS. 6A and 6B.

FIG. 12A shows a front elevation view of a lumbar spine with a coronal plane deformity (such as, for example, scoliosis).

FIG. 12B shows a front elevation view of the lumbar spine of FIG. 12A with the device installed to produce a deformity (scoliosis) correction.

FIG. 13A illustrates a top elevation view of another embodiment of the implantable device (after implantation) with the distraction mechanism positioned in the anterior aspect of the member.

FIG. 13B shows a side elevation view of the device of FIG. 13A (i.e., viewed along Direction A).

FIG. 14 illustrates an exemplary cross sectional view of a targeted disc space in the lumbar spine, illustrating various approaches thereto.

FIGS. 15-17 illustrate one embodiment of an implantable device housing, according to aspects of the present disclosure.

FIG. 18 illustrates a trajectory of approaching a space within a spinal column, according to aspects of the present disclosure.

FIGS. 19-22 illustrate various perspective views of an implantable device distraction mechanism according to aspects of the present disclosure.

FIG. 23 illustrates an implantable device housing and distraction mechanism according to aspects of the present disclosure.

FIG. 24A illustrate an exemplary embodiment of an implantable device housing and distraction mechanism in a first expanded configuration.

FIG. 24B illustrate an exemplary embodiment of an implantable device housing and distraction mechanism in a second expanded configuration.

FIGS. 25A-25B illustrate cross sectional views of a targeted disc space, illustrating one exemplary approach thereto.

FIGS. 26A-26B illustrate exemplary cross-sectional views of a targeted disc space including an exemplary surgical corridor and bone graft material.

FIG. 27 illustrates a vertebral column.

FIGS. 28-29 illustrate an exemplary method of accessing a facet space according to aspects of the present disclosure.

FIGS. 30A-30B illustrate an exemplary way of approaching a facet space, according to aspects of the present disclosure.

FIGS. 31A-31B illustrate an exemplary embodiment of screw/nut components that may be used with the implantable device of the present disclosure.

All Figures @ Copyright 2013-2020. Samy Abdou. All rights reserved.

### DETAILED DESCRIPTION

In order to promote an understanding of the principals of the disclosure, reference is made to the drawings and the embodiments illustrated therein, and wherein like numerals refer to like parts throughout. Nevertheless, it will be understood that the drawings are illustrative and no limitation of the scope of the claims is thereby intended. Any such alterations and further modifications in the illustrated embodiments, and any such further applications of the principles of the disclosed devices as illustrated herein are contemplated as would normally occur to one of ordinary skill in the art.

### Overview

In one aspect, improved devices, systems, and methods to alter vertebral alignment are described herein. Specifically, a variable height implantable device and its systems (e.g., related components) and methods of use are disclosed herein.

It will be appreciated that in a variety of disorders, the vertebral bones of a human (or other vertebrate organism) may become mal-aligned and produce, among other conditions, translational, rotational and/or angulational deformities of the spinal column. The devices and methods disclosed herein can advantageously be used in the treatment of many spinal disorders, such as, inter alia, coronal plane deformity (such as scoliosis), sagittal plane deformity (such as alternation in segmental kyphosis or lordosis), axial translation, vertical translation, spondylolisthesis, and the like.

In one implementation, a spinal segment to be surgically treated using the methods and apparatus disclosed herein includes at least a superior vertebral bone, an immediately inferior vertebral bone, and the intervening intervertebral disc space. A spinal segment comprised of two immediately adjacent vertebral bones and the intervertebral disc space disposed therebetween defines a “functional spinal unit” (FSU)—as described further below. An FSU to be surgically treated will be referred to as a target FSU and its intervertebral disc space as a target intervertebral disc space.

In one embodiment, a method of treatment includes entering the target intervertebral disc space and removing at least a portion of the viscoelastic material that comprises the natural nucleus pulposus within (at least a portion of) the intervertebral disc space. The target intervertebral disc space may be accessed using various surgical approaches (such as e.g., a direct anterior approach, an anterolateral approach, and/or a direct lateral approach, posterolateral approach, posterior approach, etc.), thereby creating one or more operative corridors at desired vertebral level(s) of the spinal column.

After removal of the viscoelastic material, the method further includes implanting a variable-height orthopedic implant into the target intervertebral disc space. The implanted orthopedic device is then actuated to vary a height of the implant in at least one aspect, and at least a portion of the implant is left in place in a substantially fixed position after the surgical procedure is complete.

The implanted apparatus enables, inter alia, customized (and heterogeneous) adjustment in distraction as between different target intervertebral disc spaces (and even different portions of the same target intervertebral space(s)). This is accomplished in one embodiment via use of one or more variable-geometry distraction mechanism used in conjunction with an implantable member, the latter which at least partly receives the former so as to create an implantable assembly that can be adjusted by the surgeon to achieve the desired geometry and spatial relationships.

### Detailed Description of the Exemplary Embodiments

Exemplary embodiments of the apparatus and methods of the present disclosure are now described in detail.

It will be appreciated that while the exemplary embodiments are described with respect to human beings, various of the methods, apparatus and systems disclosed herein may be applied to other species having a spinal structure (i.e., vertebrates).

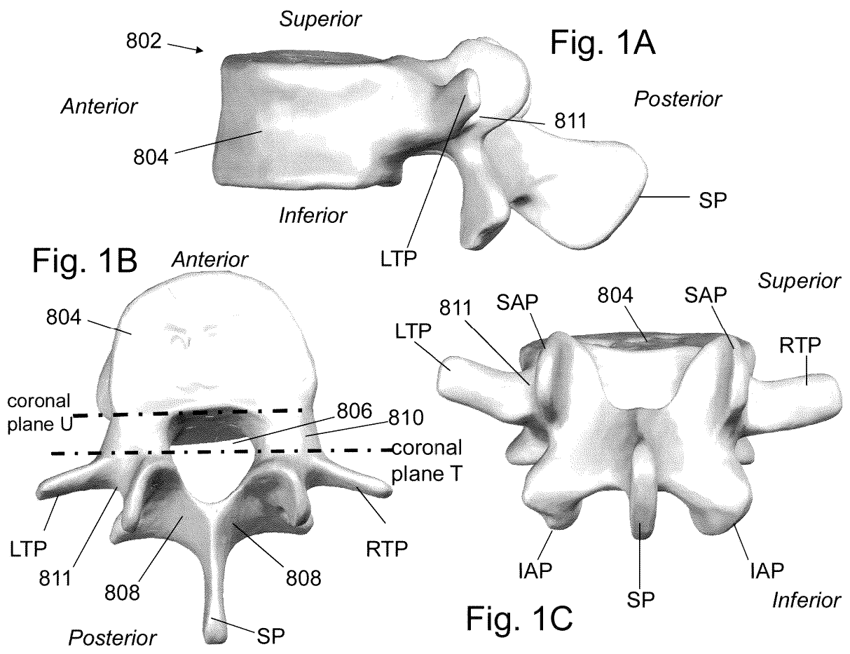

FIGS. 1A-1C show diagrammatic representations of a spinal vertebral bone 802 in multiple views. For clarity of illustration, the vertebral bone of FIGS. 1A-1C and those of other illustrations disclosed herein are represented schematically and it should be appreciated that actual vertebral bodies may include anatomical details that are not shown in these figures. Further, it is understood that the vertebral bones at a given level of the spinal column of a human or animal subject will contain anatomical features that may not be present at other levels of the same spinal column. The illustrated vertebral bones are intended to generically represent vertebral bones at any spinal level without limitation. It will be appreciated that the disclosed devices and methods may be employed at any applicable spinal level.

Additionally, the term “sagittal plane”, as used herein, refers without limitation to the plane that splits the body into left and right segments. The terms “mid-sagittal plane” or “median plane”, as used herein, refer to the plane that specifically splits the body into equal left and right halves. The term “coronal plane”, as used herein, refers without limitation to the plane that divides the body into anterior (front) and posterior (back) segments. It will be appreciated that the coronal and sagittal planes are substantially perpendicular to one another.

As can be seen in FIGS. 1A-1C, the vertebral bone 802 contains an anteriorly-disposed vertebral body 804, a centrally-disposed spinal canal 806 and a posteriorly-placed lamina 808. The pedicle segments 810 of the vertebral bone 802 form the lateral aspects of the spinal canal 806 and connect the laminas 808 to the vertebral body 804. The spinal canal 806 contains neural structures such as the spinal cord and/or nerves. A midline protrusion termed the spinous process (SP) extends posteriorly from the medial aspect of laminas 808. A protrusion extends laterally from each side of the posterior aspect of the vertebral bone 802 and is termed the transverse process (TP). A right transverse process (RTP) extends to the right from the lateral aspect of the right pedicle. A left transverse process (LTP) extends to the left from the lateral aspect of the left pedicle. A superior protrusion extends above the lamina 808 on each side of the vertebral midline, and is termed the superior articulating process (SAP). An inferior protrusion extends inferiorly below the lamina 808 on each side of the vertebral midline, and is termed the inferior articulating process (IAP).

As a brief aside, it is noted that the posterior aspect of the pedicle 810 can be accessed at an indentation 811 in the vertebral bone 802 between the lateral aspect of the SAP and the medial aspect of the TP. In surgery, it can be common practice to anchor a bone fastener into the pedicle portion 810 of a vertebral bone 802 by inserting the fastener through indentation 811 and into the underlying pedicle 810 in a posterior to anterior direction.

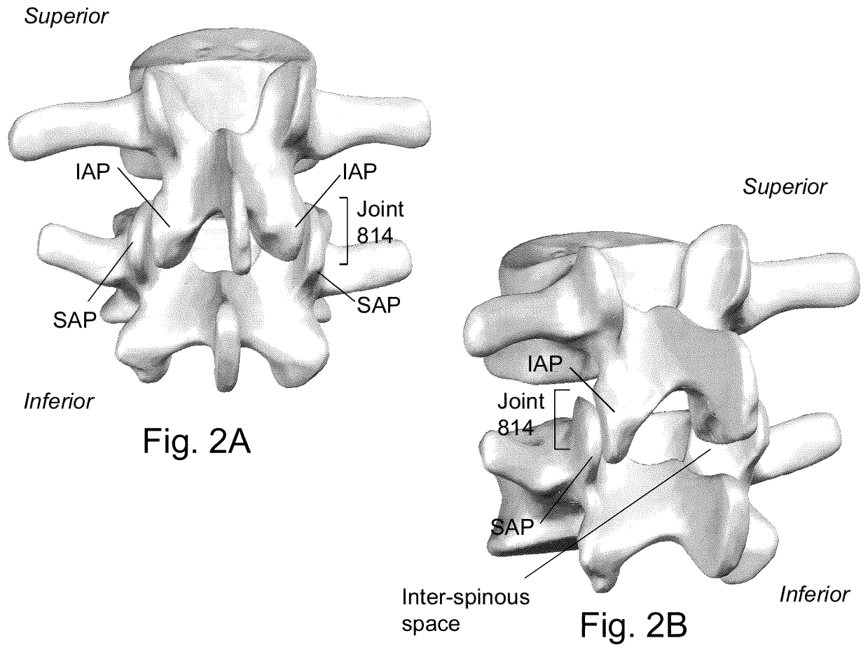

FIGS. 2A and 2B illustrate a functional spinal unit (FSU), which includes two adjacent vertebrae and the intervertebral disc disposed therebetween. The intervertebral disc resides between the inferior surface of the upper vertebral body and the superior surface of the lower vertebral body, although it is not specifically shown in the figures. FIG. 2A shows the posterior surface of the adjacent vertebrae and the articulations between them. FIG. 2B shows an oblique view. The FSU contains three joints between the two vertebral bones, with the intervertebral disc comprising the anterior joint. The posterior joints include a facet joint 814 on each side of the midline, wherein each facet joint 814 is comprised of the articulation between the IAP of the superior vertebral bone and the SAP of the inferior bone.

These illustrations and definitions of anatomical structures are known to those of ordinary skill in the art. They are described in more detail in Atlas of Human Anatomy, by Frank Netter, third edition, Icon Learning Systems, Teterboro, New Jersey, the entirety of which is incorporated herein by reference. It should be appreciated that the directional language and terms regarding orientation such as upper, lower, upward, downward etc., are used throughout merely for convenience of description and are not limiting.

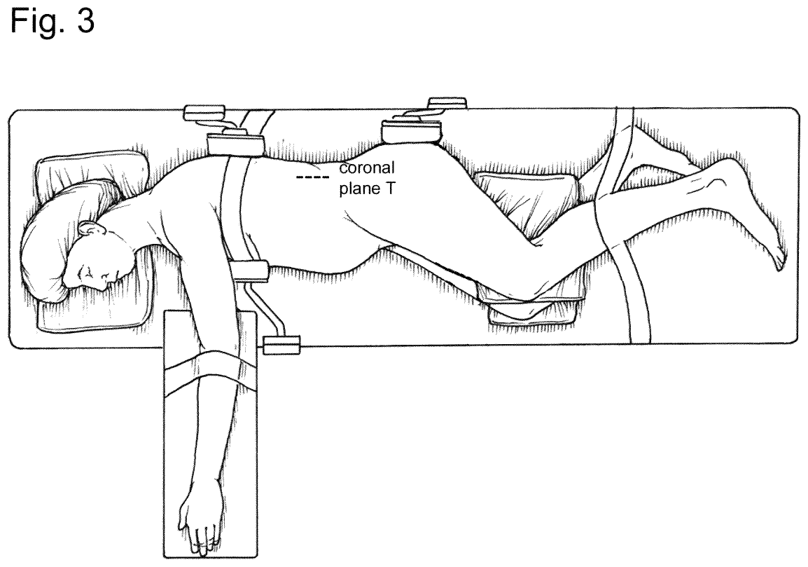

A method of device implantation is now illustrated and described. One or more FSUs is/are targeted for surgical manipulation and treatment. In preparation for surgery, the patient can be, but is not necessarily, placed in a lateral decubitus position, such as that shown in FIG. 3. It is understood that a target intervertebral disc space may be accessed using any known surgical approach, and the illustrated method serves as a non-limiting example.

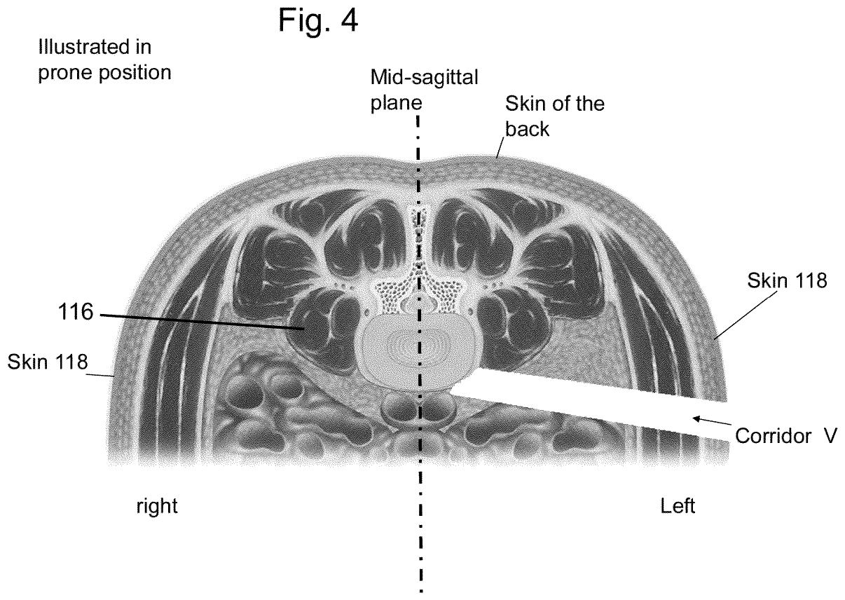

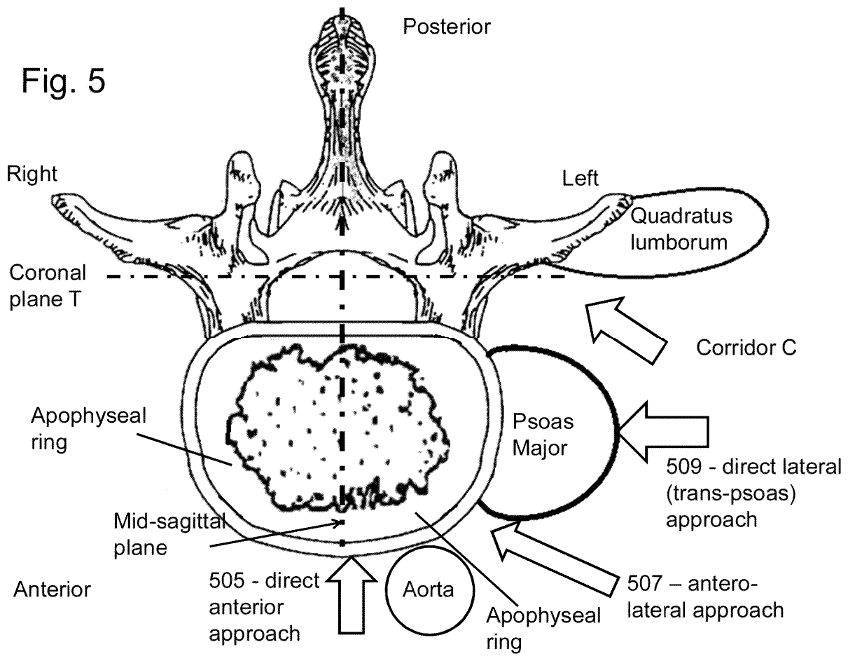

The level(s) of the spine that is to be implanted can be localized on an imaging modality (such as X-ray, CT, MRI and the like) in at least one plane. After the customary sterile preparation of the operative site, the surgeon can localize an incision point on the skin that is anterior to coronal plane T. Preferably, but not necessarily, the incision may be made immediately anterior to a coronal plane that is parallel to coronal plane T and passes through the anterior-most (tip) aspect of the target disc space. A lateral corridor “V” (FIG. 4) can be made through the skin 118 from the flank incision and taken onto the target intervertebral disc space. In the illustrated embodiments, the disc space may be entered using one or more of three locations shown in FIG. 5; i.e.: a) medial to the aorta and may comprise the midline (and/or its branches, the common iliac arteries, etc.) to form a direct anterior approach (505); b) lateral to the aorta but anterior to the Psoas Major muscle—to form an anterolateral approach (507); and c) laterally and through the body of the Psoas Major muscle—to form a direct lateral approach (509). The insertion corridor of a direct lateral approach is known to those skilled in the art as the “XLIF” procedure, among other names. See “Extreme Lateral Interbody Fusion (XLIF): a novel surgical technique for anterior lumbar interbody fusion”—Ozgur, Aryan et al. in Spine J. 2006 July-August; 6(4):435-43, which is incorporated herein by reference in its entirety.

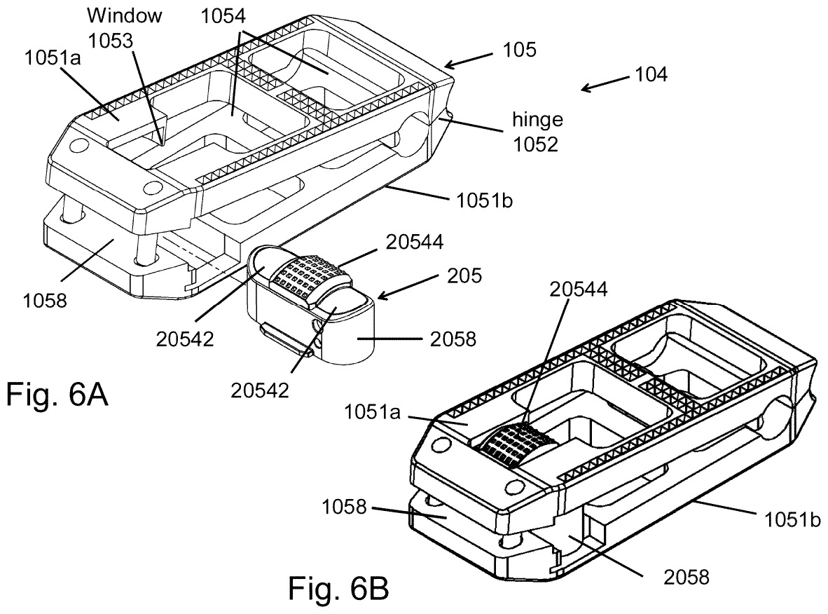

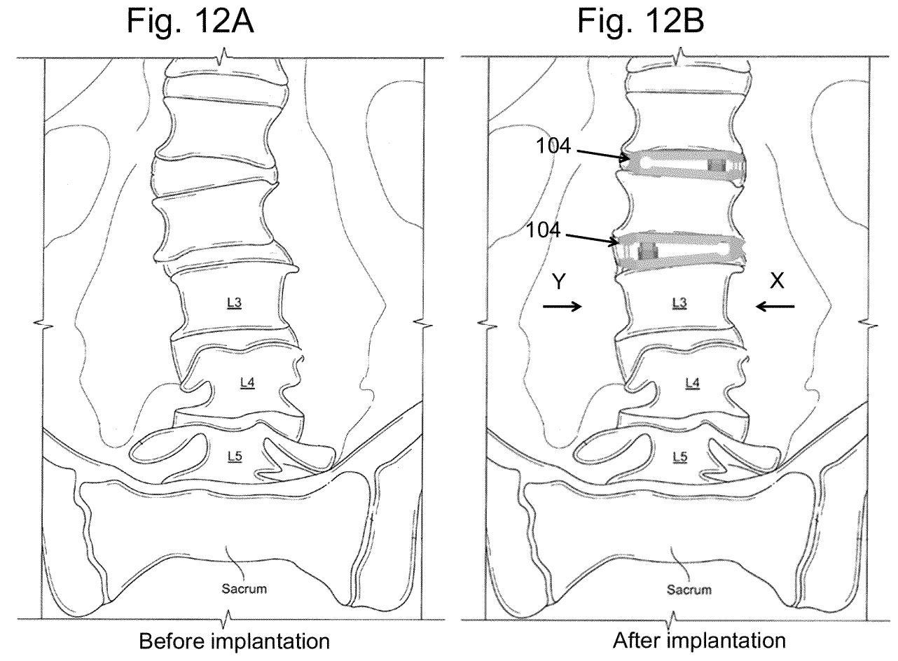

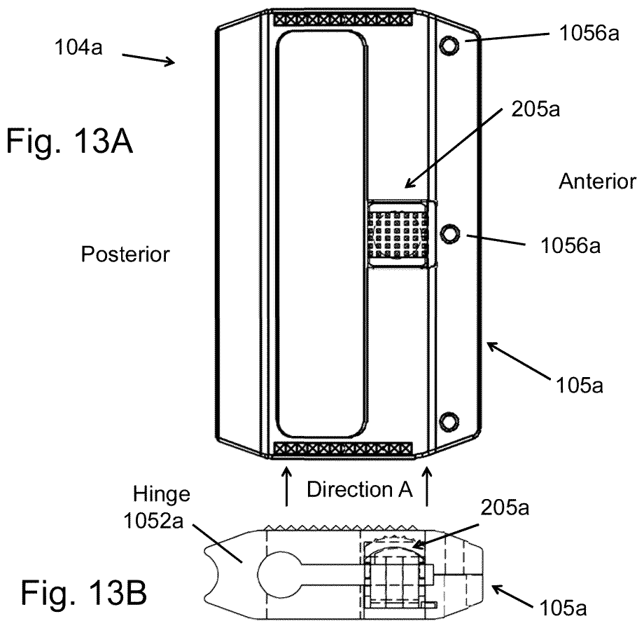

In one implementation of the method, the target intervertebral disc space is entered using the anterolateral approach 507, shown in FIG. 5, which is at least partially positioned between the lateral Aorta and the anterior surface of the ipsilateral Psoas major muscle. After removal of at least a portion of the nucleus pulposus of the target intervertebral disc space, a variable-height implant 104 (see FIG. 6A) may be placed into the disc space. Preferably, the implant 104 extends across the midsagittal plane of the disc space with one end segment positioned onto the left side of the apophyseal ring of the inferior vertebral bone and a second end segment positioned on the right side of said apophyseal ring (as subsequently shown and discussed with respect to FIGS. 12A and 12B), although it will be appreciated that other orientations and dispositions may be used (as subsequently shown and discussed with respect to FIGS. 13A and 13B). Additionally, the present disclosure contemplates that the implants 104 may be configured with a more round or oval form factor, and rotated at varying degrees (e.g., around an axis generally co-linear or parallel with the spinal column of the patient) so as to best accommodate the particular configuration of the prevailing target intervertebral disc space. Moreover, such rotations or other positional variations may be applied on a per-implant basis; e.g., wherein one target space uses one rotation/orientation, and another target space within the same patient uses another.

In one variant of the method, the superior and inferior vertebral bones of the target FSU are distracted away from one another in order to increase the vertical height of the target intervertebral disc space during implantation. Such optional distraction step may be performed with, for example, one or more distraction instrument(s) or devices that are used during surgery, and which are removed prior to the end of the procedure or after placement of the orthopedic implant(s); however, it is also recognized that dissolvable, inflatable, or other means of distracting may be utilized, other than the one or more removable distraction devices previously referenced.

In another embodiment, the method includes inserting a variable-height implantable device into a target intervertebral disc space in order to change the coronal plane alignment of the target FSU. The implant may be placed into the target intervertebral disc space using any of the known surgical approaches, such as, for example, the direct anterior approach, the anterolateral approach, and/or the direct lateral approach, discussed supra.

In one variant of the method, the implant is inserted with the first lateral side of the implant (i.e., a side of the implant that will be positioned closest to a first lateral side surface of the target intervertebral disc space) having a height that is less than or equal to the height of the second lateral side (i.e., a side of the implant side that will be positioned closest to the second lateral side surface, opposing the first lateral side surface, of the target intervertebral disc space) (see FIGS. 12A and 12B). After the implant is positioned at a desired position within the target intervertebral disc space, a mechanism of the implant is actuated so as to increase the height of one, but not both, of the first lateral side or the second lateral side of the implant. Thus in one implementation, a coronal section though the implanted intervertebral disc space will show that the implant, after actuation of the distraction mechanism, is wedge-shaped with the distance between the inferior surface of the superior vertebral bone and the superior surface of the inferior vertebral bone being greater on one lateral side surface of the target intervertebral disc space than on the opposing lateral side surface of said disc space.

In another variant, the implant comprises an anterior side (i.e., a side of the implant that will be positioned closest to the anterior midline (mid-sagittal) of the implanted intervertebral disc space) and an opposing posterior side (i.e., a side of the implant that will be positioned closest to the posterior midline (mid-sagittal) of the implanted intervertebral disc space) (see FIGS. 13A and 13B). The implant is positioned at the desired position within the intervertebral disc space, and then the mechanism within the implant is actuated so as to increase the height of one, but not both, of the anterior side or the posterior side of the implant. Thus in one implementation, a mid-sagittal section though the implanted intervertebral disc space will show that the implant, after actuation of the distraction mechanism, is wedge-shaped with the distance between the inferior surface of the superior vertebral bone and the superior surface of the inferior vertebral bone being greater at one of the anterior or posterior side sides of the implanted intervertebral disc space than at the opposing side of the target disc space.

FIGS. 6A and 6B illustrate an embodiment of the variable-height implant having a distraction mechanism according to the present disclosure. The illustrated implant 104 may, for example, comprise a first member 105 (i.e., a host member) having an “upper” element 1051a with superior surface adapted to abut the inferior surface of the vertebral bone immediately superior to the target disc space, and a “lower” element 1051b with an inferior surface adapted to abut the superior surface of the vertebral bone immediately inferior to the target disc space. The superior and inferior surfaces are connected by at least one side surface. In the present embodiment, a hinge 1052 or other mechanism for articulation is positioned at one side of upper and lower elements 1051a, 1051b and is configured to allow the inferior surface to move relative to the superior surface, thereby increasing a distance therebetween. The hinge may be a malleable or even frangible member that is connected each of the superior and inferior surfaces. Alternatively or additionally, the hinge may comprise two abutment or engagement surfaces, each disposed on one of the superior and inferior surfaces of the first member, which rotate about a common axis (such as, for example, about a central pin). Cavities 1054 open within and/or onto the superior and inferior abutment surfaces and are adapted to accept and/or house a bone forming material (including, e.g., allograft and autograft bone) in order to form a bony fusion across member 105 and between the superior and inferior vertebral bones.

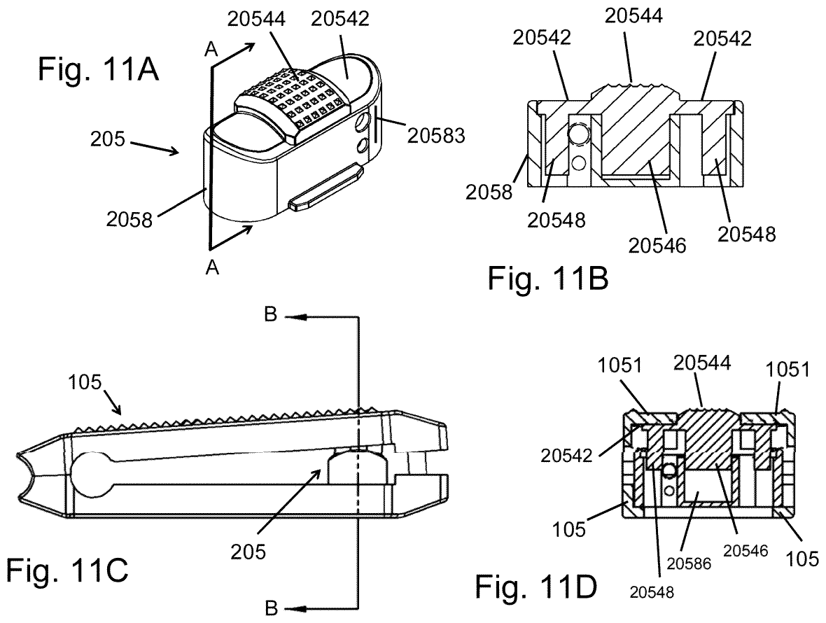

As can be seen in FIGS. 6A and 6B, the variable-height implant 104 further comprises a distraction mechanism 205 (i.e., a second expandable member), which is configured to be at least partially positioned within and/or at least partially seated within the first member 105. FIG. 6A illustrates a position for the insertion of the distraction mechanism 205 into the first member 105. After the distraction mechanism 205 is seated within the first member 105 (as in FIG. 6B), a surface 20544 extends through a window 1053 of the first member 105. A surface 20542 of mechanism 205 abuts the undersurface of upper element 1051a of the first member 105. Thus, when the distraction mechanism is seated, the surface 20542 abuts the first member 105, whereas the surface 20544 abuts the vertebral bone. This is further illustrated in the sectional view of FIG. 11B.

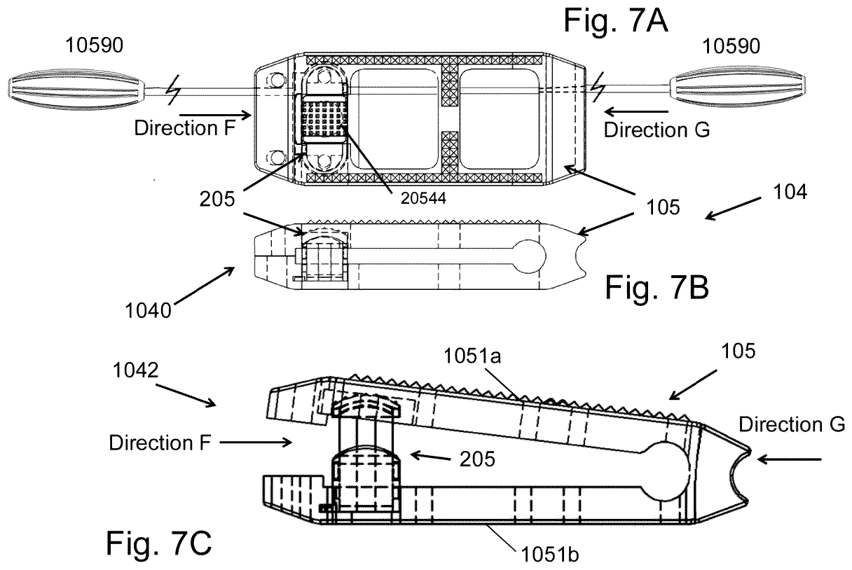

In one embodiment, upon actuation, the distraction mechanism 205 increases a height of at least one side of the first member 105. For example, upon actuation of the distraction mechanism 205, a distance between the upper and lower elements 1051a, 1051b is increased on at an expandable side 1058 (i.e., an end opposing the hinge 1052) of the first member 105. In alternate examples, the distraction mechanism may increase the height of more than one side of the first member 105. However, as illustrated herein (see FIGS. 7B and 11B), the increase in height is at least greater on one side (i.e., the expandable side 1058) than the other side(s)—so that the implant assumes a “wedge-like” configuration that can be used to realign spinal bones.

An exemplary contracted position 1040 of the implant 104 is shown in FIG. 7A, while an exemplary expanded position 1042 is shown in FIG. 7B. It will be appreciated that the implant 104 can be adjusted to assume various intermediate positions and/or positions having a greater distance between the upper and lower elements 1051a, 1051b. It is noted that, during implantation, the first member 105 is preferably inserted into the disc space in a configuration where the opposing sides are of substantially equal height (position 1040 of FIG. 7A). After implantation of the implant 104, subsequent actuation of the distraction mechanism 205 will cause the first member 105 to assume a wedge-shaped configuration (position 1042 of FIG. 7B).

In one implementation, the distraction mechanism 205 is adapted to be delivered to the surgeon as a separate device, and then installed within the first member 105 at the time of the procedure. As discussed elsewhere herein, alternatively, the distraction mechanism can be integral to the first member. Notably, the distraction mechanism 205 may be configured to be utilized with other configurations or types of implants (not shown) other than the first member 105 of the depicted implant device 104. Additionally or alternatively, the portions of the first member 105 which receive the distraction mechanism 205 can be made of a standardized configuration, such that any given distraction mechanism can be fitted with any member 105 (such as, e.g., dependent on a specific treatment or specific spinal condition). Alternatively, heterogeneous sizes and/or overall expanded/fully contracted lengths of distraction may be provided. For instance, in one such approach, three (3) sizes are provided (e.g., small, medium, and large) for different sized implant devices 104, such as for target intervertebral disc spaces (or patients) of different sizes. It is also appreciated that a single distraction mechanism 205 can be substituted (with proper adaptation of the receiving host member 105) with two or more smaller mechanisms and/or the first member 105 may be configured to receive two or more distraction mechanisms at various locations within the member, such as to permit finer adjustment of various particular portions (e.g., anterior and/or posterior portions) of the implant relative to the inferior/superior surfaces of the vertebrae which it engages when installed.

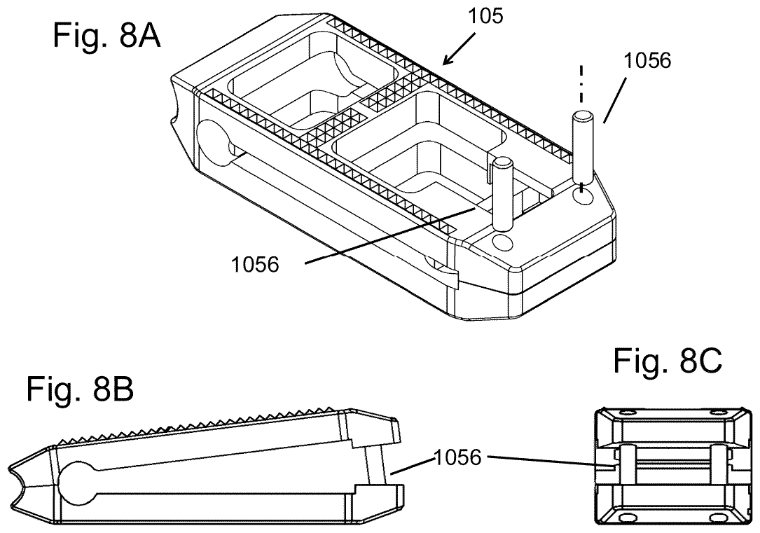

As can be seen in FIG. 8, the expandable side 1058 (i.e., the non-hinged side opposing the hinge 1052) of the first member 105 may comprise two or more segments or pins 1056 or other such mechanisms, which may be configured to, inter alia, provide added rotational stability to one or more of the implant sides. In some implementations, these pins or segments can also be configured to enable fastening or locking of the upper and lower elements 1051a, 1051b relative to one another after the distraction mechanism(s) 205 has been inserted within the first member 105, and/or after the implant 104 has been inserted into the target intervertebral space. For instance, the pins may comprise threaded or rotation-lockable couplings which engage the upper and lower elements 1051a, 1051b so as to “clamp” the variable-height implant 104 and/or the distraction mechanism 205 in place once the desired configuration (e.g., a desired height of the expandable side 1058 of the first member 105) is achieved.

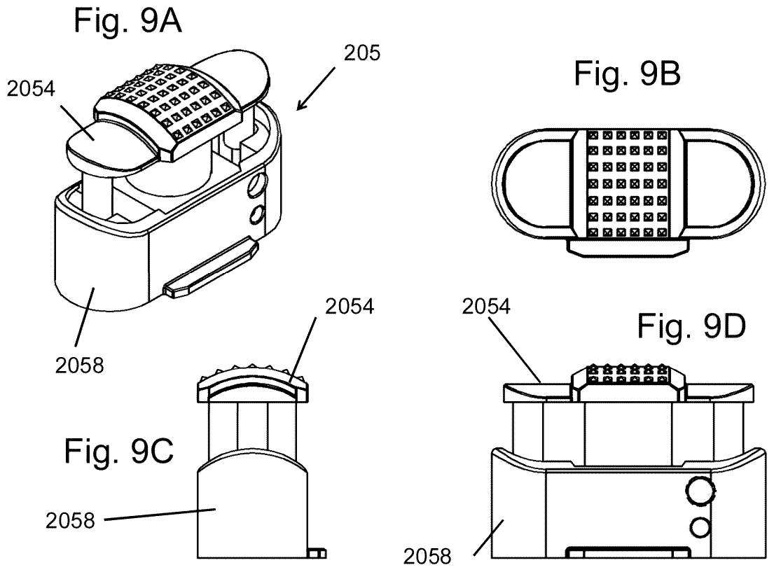

FIG. 9 illustrates multiple views of the exemplary embodiment of the distraction mechanism 205. Many embodiments of expandable interbody implants are known in the art, and these devices employ a host of differing mechanisms for device expansion and/or actuation. These mechanisms include, but are not limited to, mechanical linkages, wedges, pulleys, balloons, magnets, and/or pistons. It will be appreciated that while a fluid-based piston assembly is shown as one option (e.g., using a substantially incompressible working fluid), a compressible fluid (e.g., gas-based) working fluid may be used in certain alternate embodiments, as may a purely mechanical (i.e., non-fluidic) mechanism, such as a worm-screw drive, gear mechanism, or the like.

For example, in one embodiment, the distraction mechanism may comprise a worm screw drive or gear mechanism and an associated ridged track, which is configured to be turned (wound) via an attachable and/or insertable adjustment tool. The tool may be operated in a first rotational direction to increase a height of the distraction mechanism 205, thereby increasing a distance between upper and lower elements 1051a, 1051b. Further, in some examples, the tool may be operated in a second rotational direction to decrease a height of the distraction mechanism 205, thereby decreasing a distance between upper and lower elements 1051a, 1051b. Various exemplary mechanical (non-fluidic) mechanisms that can be adapted into the distraction mechanism 205 for use in combination with the first member 105 are shown and described in U.S. Pat. No. 7,909,870 and U.S. Patent Publication No. 2003/0163199, each of which is incorporated herein by reference in its entirety.

In another embodiment, the distraction mechanism may comprise a balloon made of either non-compliant or compliant material which may be porous or non-porous, or may include a mesh material which may be coated or lined with a porous or non-porous material. The balloon may further include a port for coupling to a source of an inflation and/or expansion medium (e.g., a gas, a liquid, a semi-solid, a gel, a liquid that hardens into a solid material, etc.) for inflating and/or expanding the distraction mechanism. The devices may further include one or more anchoring or attachment features for fixing the balloon within the first member 105. Actuation of such an embodiment of the distraction mechanism may involve inflation of the balloon with the expansion medium, wherein the act of balloon inflation provides at least part of the force needed to produce (i) the change in configuration of the structure of the housing/structure distraction mechanism (such as an increase in the height of the housing or a change in its dimension, such as length and/or width, of a segment of the housing, and/or (ii) the force needed to produce the change in configuration of the first member 105 (such as increase in a distance between the upper and lower elements 1051a, 1051b on at least one end (e.g., the expandable side 1058) of the first member 105). An exemplary balloon driven distraction mechanism that can be adapted into the distraction mechanism 205 for use in combination with the first member 105 is shown and described in U.S. Pat. No. 8,123,807, which is incorporated herein by reference in its entirety.

While the distraction mechanism 205 is illustrated as a piston-based distraction mechanism, it is understood that any distraction mechanism (such as one or more of those described supra) may be alternatively employed.

Notably, the distraction mechanism 205 may also be configured for reversible mating with the first member 105 intra-operatively, when handled by the surgeon. That is, the distraction mechanism may have one or more degrees of chirality or “handedness,” such that (i) it can be inserted in one orientation, and also in the opposite orientation (e.g., rotated 180-degrees from the first orientation around an axis generally parallel to the patient's spine), and/or (ii) can be inverted such that its otherwise superior surface can function as its inferior surface, or vice versa). The foregoing approaches advantageously mitigate the implanting surgeon “fumbling” with the mechanism 205 during surgery to achieve the proper orientation, and more importantly can avoid any instances where the mechanism 205 is installed in an improper orientation within the host member 105. To that end, the mechanism 205 and its host member 105 may also include mechanical alignment features such as keys, indexing, pins, etc. such that it can only be inserted one way. Additionally or alternatively, the distraction mechanism may be intra-operatively removable from the first member 105, when handled by the surgeon. That is, in an example where the pins 1056 provide clamping or locking of the first member 105 after distraction caused by the distraction member 205, the distraction member may be reversibly distracted (i.e., its height decreased) and removed from the first member prior to completion of the surgical procedure, while the first member retains its wedge-like configuration.

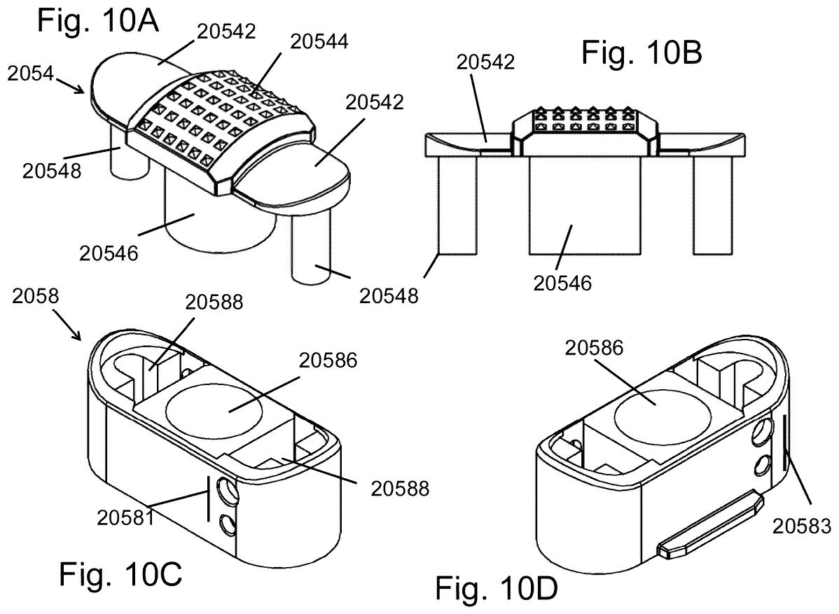

FIG. 10 illustrates an exemplary configuration of the constituent members 2054 and 2058 of the distraction mechanism 205. In the exemplary configuration, a piston 20546 is received within a cavity 20586 of the member 2058. Side protrusions 20548 are each received within cavities 20588 and function to provide, inter alia, alignment and rotational stability to the distraction mechanism 205. The member 2054 has surfaces 20542 that abut the undersurface of the superior surface 1051a of the first member 105, whereas surface 20544 extends through the window 1053 (see FIGS. 6B and 11B). In certain embodiments, multiple sub-segments (such as sealants/gaskets, O-rings, etc.), which may be used with the described piston-based approach, are known components of piston devices, and are not repeated here for diagrammatic simplicity. Note that in one implementation, the distraction mechanism 205 contains multiple apertures 20581 and 20583 that allow filling and/or bleeding of the working fluid from the piston chamber. See also, e.g., U.S. Patent Application Publication No. 2007/0093901, herein incorporated by reference in its entirety, which describes the exemplary use of pistons in the manufacture of an expandable interbody implant, such as may also be used within the distraction mechanism 205 described above.

Advantageously, the exemplary embodiment of the distraction mechanism 205 is configured such that it can be actuated from at least two separate sides. That is, the aperture(s) 20583 is/are formed within a first surface and the aperture(s) 20581 is formed within a second surface of mechanism 205. Hence, different directions of approach can be used to actuate the distraction mechanism 205. In one implementation, the distraction mechanism 205 is configured to actuate whether the actuation tools approach (e.g., are attached to it) from either or both of Direction F or Direction G of (FIG. 7B); i.e., from one side of the device 104 or the other. For example, either or both of handles 10590 illustrated in FIG. 7A can be used actuate the distraction mechanism 205. The handles 10590 can also be provided regardless of the nature of the distraction mechanism used (mechanical linkages, wedges, pulleys, balloons, magnets, pistons and the like), such that the device may be actuated whether the first member 105 is approached from one side or the opposing second side. FIGS. 11A and 11B illustrate sectional views of the exemplary embodiment of the distraction mechanism 205 alone (FIG. 11A), and with the distraction mechanism 205 positioned within the first member 105 (FIG. 11B).

FIGS. 12A and 12B illustrate an example of a spinal deformity correction that may be achieved with use of one or more aspects of the disclosed invention(s). In one implementation, a surgeon may be given a kit comprising the first member 105, the distraction mechanism(s) 205, and handling or installation instruments for, at least, positioning said members, and/or actuation of the distraction mechanism(s) 205 (such as handle(s) 10590). The patient can be prepared and a surgical procedure performed by the surgeon as described above.

### Exemplary Applications

In one embodiment, a variable-height implant may be configured to form a lateral wedge configuration and be used to alter the spinal alignment of a target functional spinal unit in the coronal plane in order to treat coronal plane deformities such as, e.g., scoliosis. FIG. 12A shows a lumbar spine with a coronal plane deformity (such as, for example, scoliosis). Application of the variable-height implant 104 (comprising both the first member 105 and the distraction mechanism 205) via one or more of the surgical procedures described above produces the deformity (scoliosis) correction shown in FIG. 12B. Note that the implant 104 advantageously may be implanted from either side of the spine (along either of directions “X” or “Y”; however, implantation of the depicted inferior (lower) implant, in the direction X, would be impossible under the prior art. For example, an implant having a fixed wedge shape (i.e., having a first side of a greater height than a second opposing side) is implantable from only one side of the target intervertebral disc space. Specifically, the foregoing fixed wedge implant can only be inserted into a disc space leading with the second opposing side (the side having a smaller height) and trailing with the first side (having a greater height).

Returning to FIG. 12B, in one exemplary application of the present invention, the variable-height implant 104 is passed into the target disc space along direction X, while the implant is in the collapsed configuration 1040 (FIG. 7A) and the leading edge (the edge that enters the disc space first) is of lesser or equal height to the trailing edge of the implant (i.e., the edge that enters the disc space last). In an alternate exemplary application, the variable-height implant 104 is passed into the target disc space along direction Y, while the implant is in the collapsed configuration 1040 (FIG. 7A). In either application, the distraction mechanism 205 may be actuated after placement of the (assembled) implant 104 into the target disc space.

It will be appreciated, however, that in the alternate exemplary application, the variable-height implant 104 may passed into the target disc space along direction Y, while the implant is in the expanded configuration 1042 (FIG. 7B) or a partially expanded configuration, after full or partial actuation of the distraction mechanism 205.

It will be further appreciated that the assembly of the implant device 104 may be conducted after the first member 105 is inserted into the target disc space in some cases. Specifically, in one variant, the first member 105 alone is inserted in the direction X into the target space, and then subsequently, the surgeon accesses the disc space (and implant host member 105) via a different approach. In another variant, the first member 105 alone is inserted via an anterior approach (having a larger surgical corridor) into the target disc space, and then subsequently, the surgeon accesses the disc space (and the first member 105) via a posterior approach (having a smaller surgical corridor). In each of the foregoing variants, the distraction mechanism 250 is initially in its completely compressed state (i.e., smallest possible vertical profile, such that the surgeon can slide the mechanism 250 into e.g., a lateral groove formed on the first member 105 (not shown), or even separate the upper and lower elements 1051a, 1051b far enough while in the disc space such that the distraction mechanism can be inserted therebetween (and subsequently expanded as described supra).

As previously discussed, a variable-height implant may be configured to form an anterior to posterior wedge configuration and be used to treat sagittal plane deformity such as, e.g., abnormal lordosis and/or kyphosis—as shown in the implant 104a of FIGS. 13A and 13B. This device embodiment may be used to correct sagittal plane deformity, and would be employed to perform the “second method embodiment” discussed above. (It is understood that whether used to correct coronal or sagittal plane deformity, the implant may be delivered into the disc space using any known surgical approach for device implantation. Specifically, approaches 505, 507 and/or 507 of FIG. 5, may be employed). FIG. 13A illustrates the implant 104a when viewed from above (for example, after implantation) including a distraction mechanism 205a positioned in the anterior aspect of a first member 105a (including three pins 1056a). FIG. 13B shows a side view—as would be seen with the device of FIG. 13A is viewed along direction A. It will be appreciated that the implant 104a may be used with any of the surgical procedures and may include the variations discussed herein. It will be further appreciated that the variable height implant 104a (in use) can be included in a kit with and/or used in combination with the implant 104 during a surgical procedure.

In yet another embodiment, a variable-height implant may be configured to form a wedge configuration used to treat translational deformities such as e.g., anterior/posterior or lateral spondylolisthesis.

It will be appreciated that the flexibility in use and configuration, as well as the modularity, of the first members 105, 105a and the distraction mechanisms 205, 205a are advantageous over prior implantation devices and methods. In other words, the configuration of the implant assembly and the method of implantation can be adapted by a surgeon to be spinal condition and/or patient specific. Such specificity may enable a surgeon to map out and prepare the implant components and surgical strategy prior to the implantation procedure. Alternatively, the surgeon can respond “on the fly” as a surgery progresses, if necessary, to provide a “best fit” implant configuration and implantation process (particularly in response to unforeseen issues that may unexpectedly arise and/or in treatment of especially complex spinal conditions). For example, any of the first members 105, 105a or the distraction mechanisms 205, 205a can be utilized as a free standing implant (e.g., where the distraction mechanism is implanted in the target disc space and actuated without prior insertion into the first (host) member, where the first member is inserted without a distraction mechanism and does not require distraction after implantation, where the first member is implanted with and distracted by the distraction mechanism, which is subsequently removed from the first member, etc.), and/or they can be used in combination (e.g., where the distraction mechanism is fitted within and is actuatable within the first member, as described supra). In other examples, various implant assemblies can be used in combination within the same target FSU or other target FSUs (e.g., adjacent FSUs) to treat complex spinal curvature conditions. The various implant assemblies can be inserted from any desired approach and/or entry point of the target FSU (such as those discussed supra).

### Exemplary Embodiment of an Implant

The segment to be surgically treated is comprised of at least a superior vertebral bone, an immediately inferior vertebral bone and the intervening intervertebral disc space. (A segment comprising two immediately adjacent vertebral bones and the intervertebral disc space between them is called a “functional spinal unit” (FSU)—as will be discussed further below. The functional spinal unit to be surgically treated will be referred to as the target functional spinal unit and its intervertebral disc space as the target intervertebral disc space.) In an embodiment, the target intervertebral disc space is entered and at least a portion of the viscoelastic material that would comprise the natural nucleus pulposus is removed. The target intervertebral disc space may be accessed using any known surgical approach—including, but not limited to, anterior, anterolateral, lateral, posterior-lateral or posterior approaches.

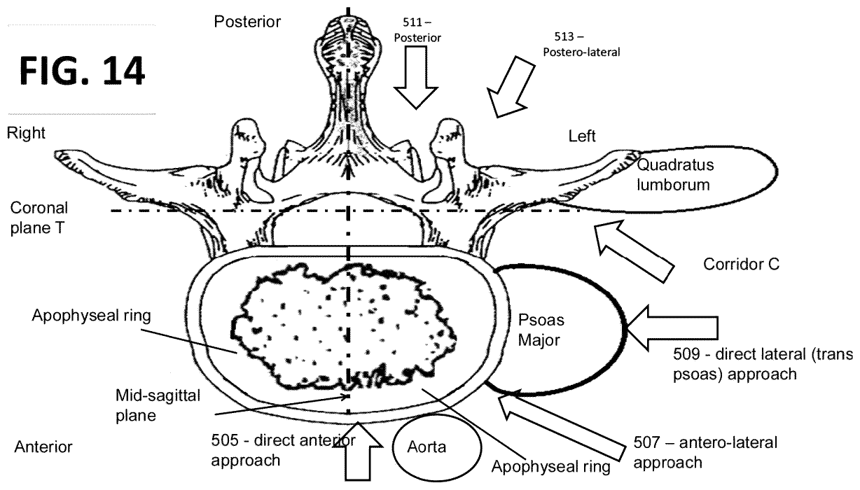

Referring to FIG. 14, in anterior, anterolateral or lateral approaches, the target disc space may be entered through the abdominal cavity using at least one or more of the three following locations: a) 505, medial to the aorta and may comprise the midline (and/or its branches, the common iliac arteries, etc) to form a direct anterior approach; b) 507, lateral to the aorta but anterior to the Psoas Major muscle—to form an anterolateral approach; and c) 509, laterally and through the body of the Psoas Major muscle—to form a direct lateral approach (FIG. 14). After removal of the viscoelastic material, an orthopedic implant may be implanted into the target intervertebral disc space using the same surgical corridor and then left in place after surgery is complete. Additionally, the superior and inferior vertebral bones may be distracted away from one another in order to increase the vertical height of the target intervertebral disc space. The optional distraction step may be performed with distraction instrument(s) that are transiently used during surgery and then removed prior to the end of the procedure or by said orthopedic implant(s) that is positioned during surgery and left in place.

The insertion corridor of approach 509 is known to those skilled in the art as the trans-psoas approach, direct lateral (DLIF), Extreme Lateral Interbody Fusion (XLIF) approach, among other names. (See “Extreme Lateral Interbody Fusion (XLIF): a novel surgical technique for anterior lumbar interbody fusion.” By Ozgur, Aryan et al. in Spine J. 2006 July-August; 6(4):435-43, which is hereby incorporated by reference in its entirety.)

The insertion corridor of approach 511 and 513 is performed using a posterior surgical approach that is posterior to coronal plane T (FIG. 3). These approaches do not require traversing the abdominal cavity but provide limited access windows through which the implant may be positioned into the intervertebral disc space since the spinal canal, and the nerves contained therein, limit the corridor size.

In a first embodiment, an implantable device is inserted into an intervertebral disc space in order to change the alignment of the spinal segment containing the implanted intervertebral disc space. The implant may be placed into the intervertebral disc space using any of the known surgical approaches, such as, for example, approach 505, 507 509, 511 and/or 513 discussed above.

In one aspect of the disclosure, the implant comprises at least two members, the first being a housing (which may comprise more than one segment), the housing configured to abut each of the vertebral bones superior and inferior to the target intervertebral disc space into which the implant is positioned. In an embodiment, the first member is not a balloon but is comprised of a solid material. In one variant, the solid material is malleable and adapted for implantation into a human or animal subject. In another embodiment, the first member is at least partially comprised of a balloon.

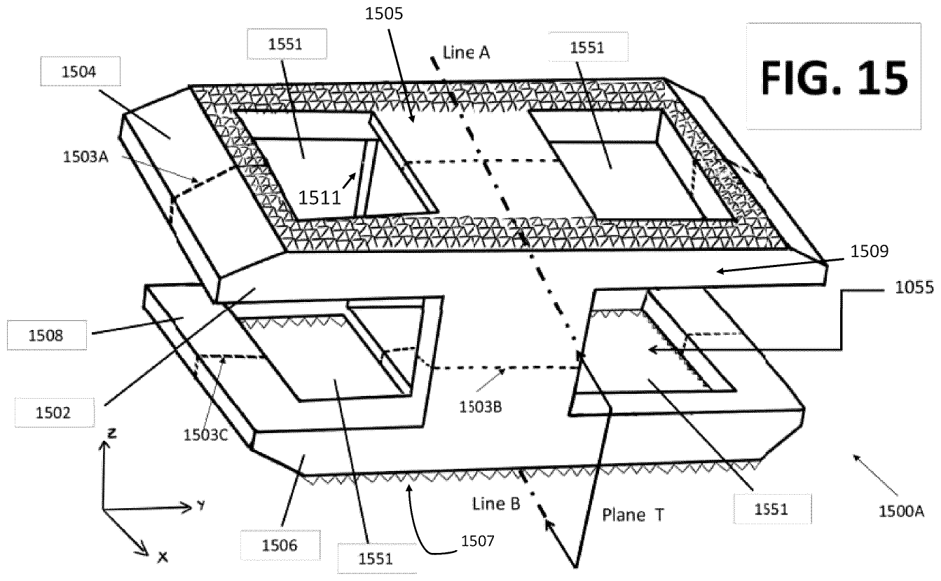

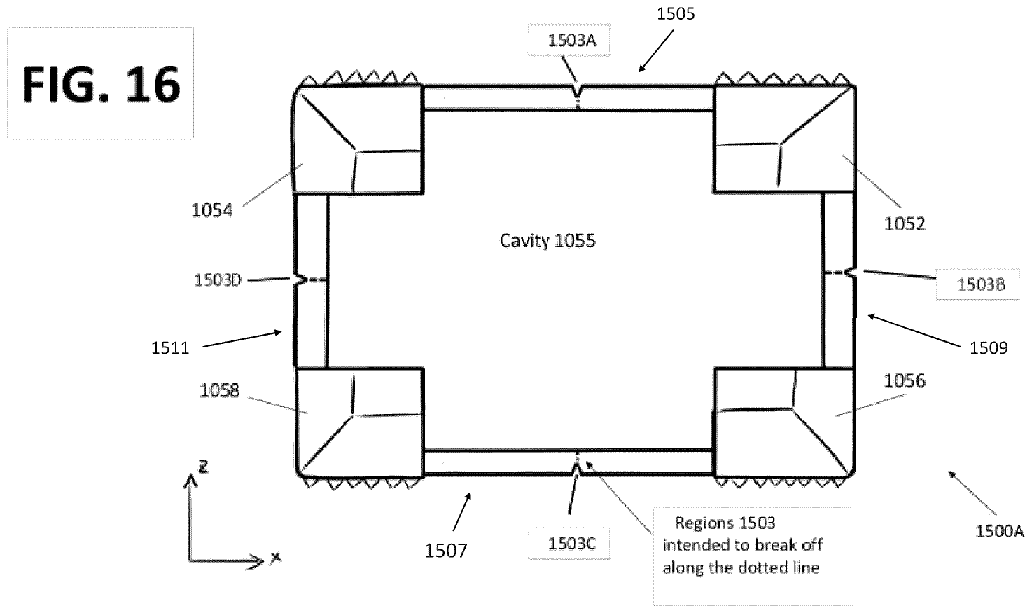

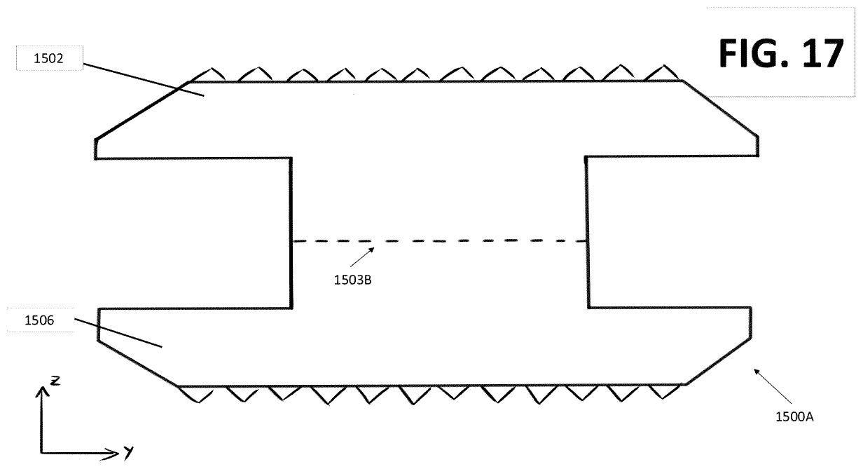

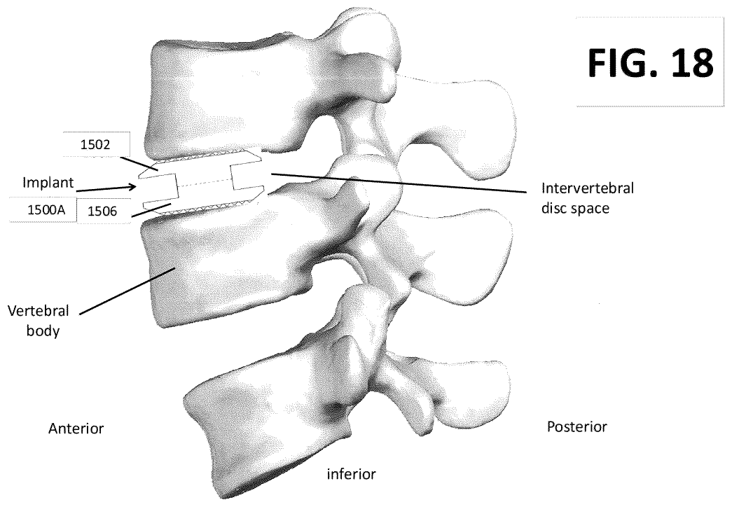

One exemplary embodiment of housing 1500A is illustrated in FIGS. 15-17. In an embodiment, the housing 1500A may be comprised of a top surface 1505, a bottom surface 1507, a first side surface 1509, and a second side surface 1511 as shown in FIGS. 15 and 16, at least a first segment 15 configured to at least abut an inferior aspect of the superior vertebral bone and second segment configured to at least abut a superior segment of the inferior vertebral bone. The housing 1500A of FIG. 15, for example, may comprise two segments 1502, 1504 that may be implanted to about the inferior aspect of the superior vertebral bone. It may also comprise two segments 1506 and 1508 that may be implanted to abut the superior aspect 20 of the inferior vertebral bone. Housing 1500A is illustrated within an intervertebral disc space in FIG. 18.

A section through housing 1500A is shown in FIG. 6. The section is shown at plane T of FIG. 15. (Plane T is comprised of a plane containing parallel, but not co-linear, Line A and Line B.) Two or more of segments 1502, 1504, 1506 and 1508 may be held together during the advancement of housing 1500A into the target intervertebral disc space. After implantation, one or more segments may be detached from one another and/or distracted apart so that the overall surface area of the outer aspect of implant 1500A and/or the volume contained within the outer aspect of housing 1500A is enlarged after said detachment and/or distraction. This may be accomplished by increasing a distance between at least two segments of housing 1500A so as to increase the overall length, height and/or width of the housing 1500A.

The segments of 1502, 1504, 1506 and 1508 may be held together during the advancement of the housing 1500A into the target intervertebral disc space using any means known or yet to be known for holding the segments in proximity to one another during the step of implant advancement. These means include, but are not limited to, clips, friction members, screws/nuts, ratchets, tethers (whether malleable or non-malleable) and the like. In addition to, or instead of, a means for direct attachment to one another, the segments may be held together by their common attachment to a member outside of the housing 1500A itself—such as, for example, the placement instrument(s) used for advancing housing 1500A into the target intervertebral disc space.

In an embodiment, the segments of housing 1500A may be held together by one or more weakened regions 1503 (1503A/1503B/1503C/1503C shown in FIG. 16), as show in FIGS. 15 and 16. Four of the weakened regions 1503 are shown in FIG. 16. At least four more weakened regions 1503 in the housing 1500A are not shown in the cross section of FIG. 16. The weakened regions 1503 may comprise, for example, areas of decreased material thicknesses, as shown, and the thicknesses may be dimensioned to control the force at which the weakened regions 1503 break and the segments separate from one another. In addition to thickness, the force(s) needed to cause segment separation may be dependent on, among other factors, on the material from which housing 1500A is manufactured. The force(s) needed to cause segment separation may be pre-set at the time of manufacturing and the housing 1500A may be made and provided to the end-user (such as, for example, surgeon) in various embodiments from which the end user selects the implant that may best fit their need for the target disc space. It is contemplated that various break off segments within a single housing 1500A may be pre-set so as to require different force levels/thresholds to cause the different pairs of segments to separate. In one embodiment, the manufacturer may pre-set different force requirements for segment separation of housing 1500A members that are intended for use in different levels (cervical, thoracic and/or lumbar) of the spinal column. These features allow the end user (surgeon) to customize the housing 1500A to the anatomy of the target disc space. In one variant, the force level required for segment separation within a housing 1500A may be pre-set to allow a desired dimension (length, width and/or height) of the implant to preferentially enlarge to a greater extent than another dimension. In one variant, the force level required for segment separation may be re-set so as to produce a particular order of segment separation wherein, for example, the length, width and/or height are increased in a particular desired order.

For example, the housing 1500A can be configured such that a first force level/threshold is required to separate segments in a first direction and a second, greater force level is required to separate segments in a second direction. In one implementation, the housing 1500A may be configured to first separate along top and bottom weakened regions (from a first force level), and then separate along side weakened regions (from a second, greater force level). In another implementation, the housing 1500A is configured to separate along side weakened regions first (from a first force level) and top/bottom weakened regions second (from a second, greater force level). A surgeon may: (i) choose to expand the housing in only one direction (length, width, or height) by applying only a first level of force and not reaching the second level of force or (ii) choose to expand the housing in two directions (two of length, width, or height) in a predetermined order by increasing the applied force to the first level and then to the second level.

In one variant, the weakened regions 1503 are separated into two or more sets, such that weakened regions in the same set are configured to break at the same level of force. In one implementation, weakened regions formed along opposite sides of the housing are grouped in the same sets. In another implementation, weakened regions formed in adjacent sides of the housing are grouped in the same sets. In one implementation, each weakened region is configured to break at a different, predetermined level of force. This would allow the housing 1500A, for example, to expand into a wedge shape by breaking only one weakened region. In another example, two or more adjacent weakened regions of the housing 1500A could be separated, leading to a lopsided expanded housing.

In various exemplary embodiments of the disclosure, the weakened regions 1503 of the housing 1500A are located along central lines of housing 1500A (as shown in FIG. 16). In other embodiments, at least some of the weakened regions 1503 are located closer to some segments of the housing 1500A than others. In another embodiment of the disclosure, the weakened regions 1503 of the housing 1500A are formed along straight lines. In other embodiments, at least some of the weakened regions 1500 may be formed along curved lines. In one embodiment, the weakened regions 1503 are aligned to the sides of the housing 1500A (i.e., are parallel with one or more edges of the housing). In another embodiment, at least some of the weakened regions are formed at an acute or obtuse angle to at least one of the housing sides.

The second member of the implant 1500 may be used as a distraction member to separate the segments of housing 1500A. Alternatively, or in addition, the housing 1500A may be distracted by the (non-implanted) instrumentation used for the insertion of the housing 1500A at the target disc space. In an embodiment of the latter, the second member may be then used to retain the separated segments of housing 1500A in the desired position/configuration relative to one another after removal of the (non-implanted) instrumentation used for the insertion of the housing 1500A. Alternatively, the second member may be used to further distract the segments of housing 1500A so as to provide greater customization to the final implant configuration.

The second member may be of fixed dimensions or a it may comprise an expandable distraction member that may be actuated to assume different dimensions.

Many embodiments of expandable distraction members are known in the art and these devices employ a host of differing mechanisms for device expansion. These mechanisms include, but are not limited to, mechanical linkages, wedges, pulleys, balloons, magnets, pistons and the like. US Pat. Application No. 2007/0093901, among others, describes the use of pistons in the manufacture of an expandable interbody implant.

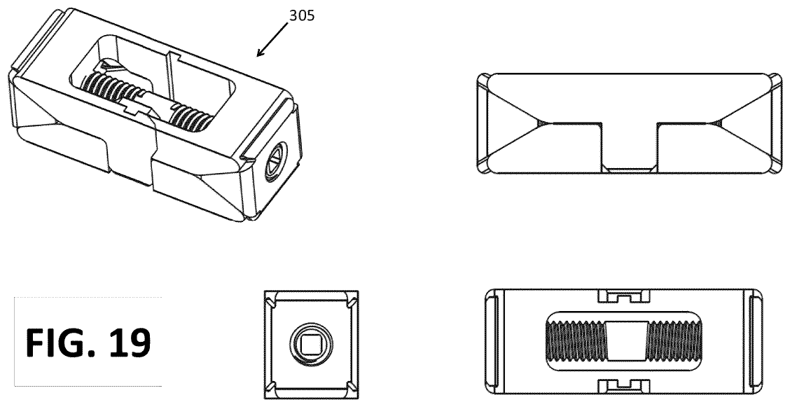

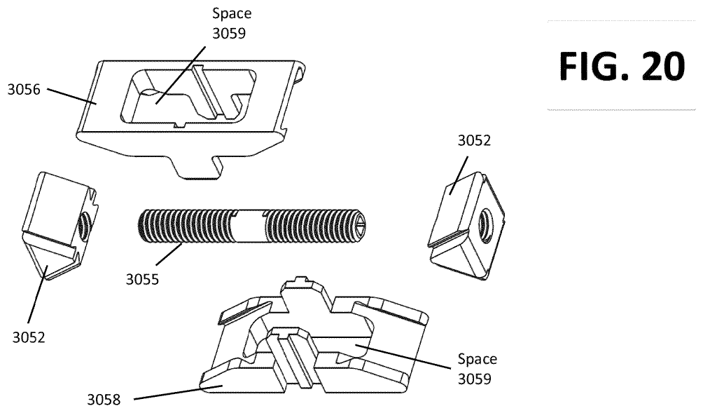

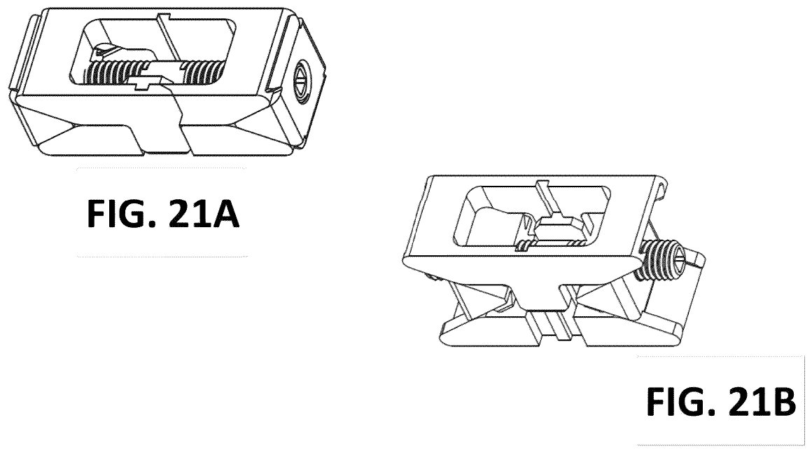

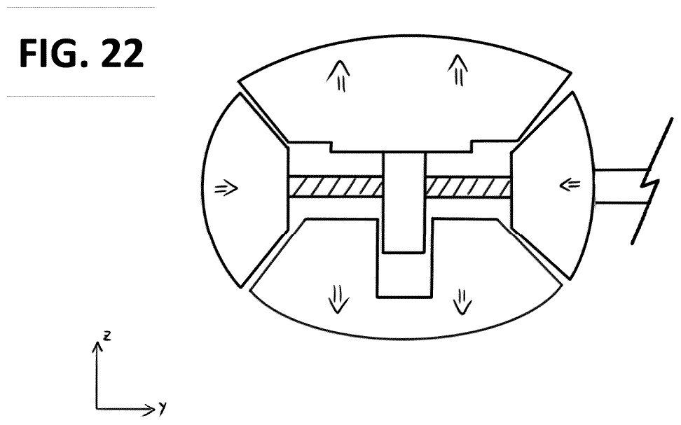

It is understood that any of the expandable distraction members recited above may be used. By way of non-limiting example, FIGS. 19 through 21 illustrate an example of a second member that comprises expandable distraction member 305. FIG. 19 shows a perspective view as well as three orthogonal views. An exploded view is shown in FIG. 20. A collapsed view is shown in FIG. 21A and an expanded view is shown in FIG. 21B. The expandable member 305 comprises wedge member 3052 (which may be similar or differing design, slop, size, etc.), locking screw 3055 and abutment surfaces 3056 and 3058. A space 3059 may be contained at least partially in distraction member 305 and configured to house a bone forming substance so as to form a fusion between the vertebral bones superior to and inferior to the target disc space. FIG. 22 shows a schematic view of the distractive motion occurring within distraction member 305 by turning locking screw 3055 in a first direction. It is understood that rotation of screw 3055 in the first direction produces the distracted position shown in FIG. 21B, whereas rotation of screw 3055 in the opposite direction reverses the motion of FIG. 22 and returns member 305 to the collapsed configuration of FIG. 21A.

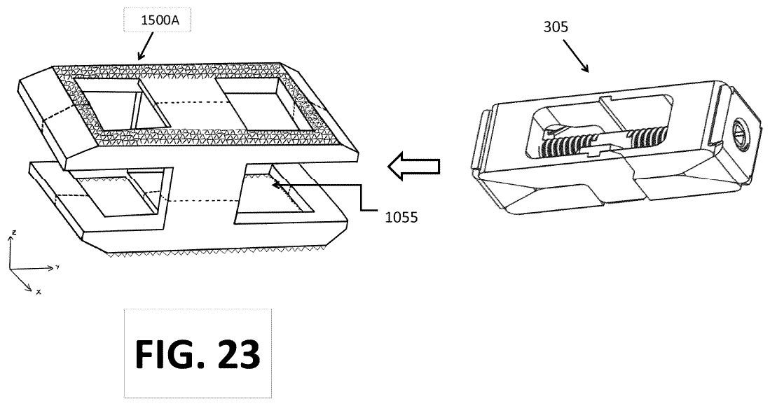

In use, housing 1500A is advanced into the target disc space as shown in FIG. 18. Distraction member 305 is then advanced into cavity 1055 of housing 1500A as show in FIG. 23. Member 305 may be advanced into cavity 1055 in the fully collapsed position. It may be of sufficiently small so as to exert insufficient force to separate the segments of housing 1500A during its advancement into cavity 1055. Alternatively, member 305 may be advanced into cavity 1055 in a partially distracted state so as to separate the segments of housing 1500A during its advancement into cavity 1055. In another embodiment, member 305 may by sufficiently large in the fully collapsed state so as to separate the segments of housing 1500A during its advancement into cavity 1055.

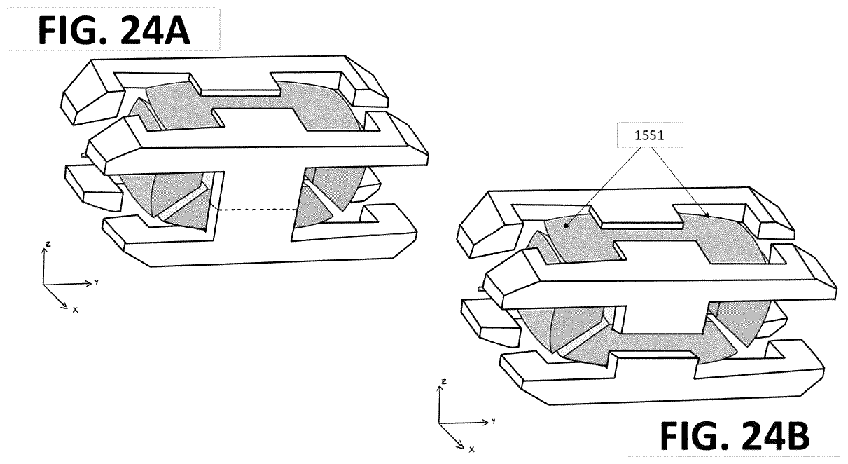

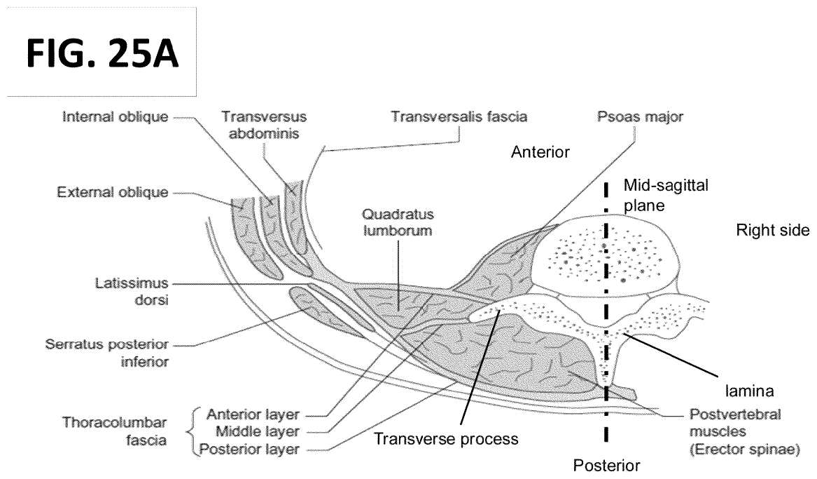

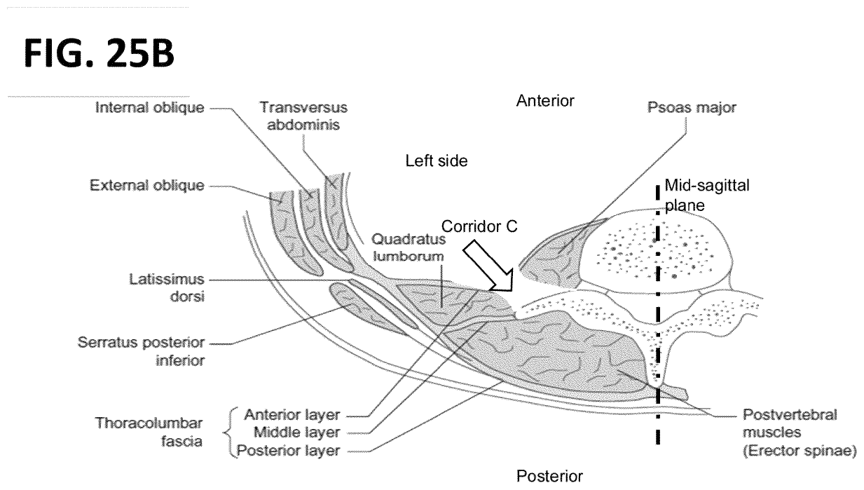

In an embodiment, after member 305 is seated within cavity of 1055 of housing 1500A, screw 3055 may be actuated to produce expansion of member 305 and provide sufficient force to break at least one region 1503 that had not been broken by the advancement of member 305 into cavity 1055. As discussed above, the specifics of which region 1503 is broken, and in what order, will depend on the specific design of the regions 1503. FIGS. 24A and 24B illustrate the breaking of different regions 1503. Finally, apertures 1551 allow the bone forming material to form a fusion across the housing 1500A and between the superior and inferior vertebral bones that surround the target disc space.

### Vertebral Bone Fixation

Many surgeons employ bone screws or other fasters to immobilize the vertebral bones adjacent to the target disc space while the disc space heals and the bony fusion solidifies. These bone screws/fasters provide greater fixation when placed into the posterior aspect of the vertebral bones. Hence, when employing the posterior corridor (511) or the posterolateral corridor (513) (FIG. 14), the bone screws/fasters can be placed without the need for additional surgical exposure.

When using the intra-abdominal approaches (approach 505, 507 and 509), insertion of the bone screws/fasteners will require an additional surgical exposure to access the posterior elements of the vertebral bone. A novel approach has been developed wherein the same incision/corridor used for approach 505, 507 or 509 can be extended to access the posterior elements of the vertebral bone. In that approach, at least a portion of the surgical corridor (corridor C of FIG. 3) may be oriented so as to extend through the anterior layer of the thoracolumbar fascia. (A full description of the anatomy of the thoracolumbar fascia is contained in: The thoracolumbar fascia: anatomy, function and clinical considerations. Willard F. H., et al. J Anat. 2012 December; 221(6): 507-536. The article is hereby incorporated by reference in its entirety.) Development of this surgical corridor (corridor C) is illustrated in FIGS. 3, 14 and 15. As shown, corridor C is developed between the posterior aspect of the ipsilateral Psoas Major and the anterior and medial aspect of the ipsilateral Quadratus Lumborum muscle. While corridor C is intended to substantially extend between these two muscles, it may contain at least a segment of each of them. Corridor C is thereby intended to be anterior to the anterior surface of the ipsilateral transverse process of the inferior vertebral bone of the target FSU and posterior to at least the posterior one half of the ipsilateral Psoas major muscle when the latter is measured in a sagittal plane that traverses it. In the superior lumbar spine, the Psoas is usually a small muscle and it increases in size as it extends inferiorly. In some segments of the spine, such as the thoracic spine, the Psoas major muscle is not present at all. Where the muscle is absent, it is understood that Corridor C is defined by its relationship to the ipsilateral transverse process and not by its relationship to the Psoas muscle.

Preferably, the anterior layer of the thoracolumbar fascia is traversed by corridor C. Dissection may be continued through corridor C in order to traverse coronal plane T in an anterior to posterior direction. In this way, the ipsilateral transverse processes of the vertebral bones of the target functional spinal unit may be reached. Similarly, segments of the target functional spinal unit that are positioned posterior to coronal plane T may be accessed through corridor C—as well be discussed further below.

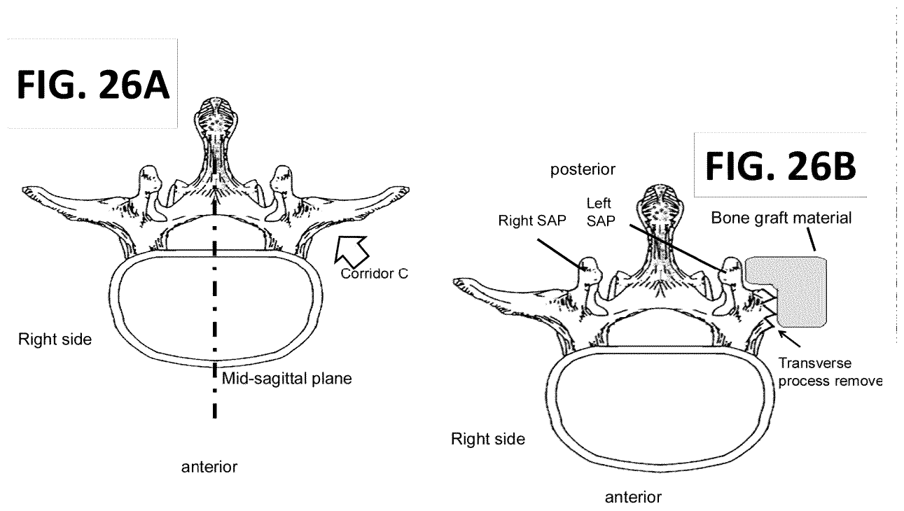

If desired, the ipsilateral transverse process of either said superior or inferior vertebral bone of the target functional spinal unit may be removed through corridor C (FIGS. 26A and 26B). The harvested transverse process bone may be used as autograft bone for a fusion procedure that is concurrently performed at the same operation. That is, the preceding steps constitute a method for removal of a transverse process of said target functional spinal unit. In this method, an intra-abdominal (and, preferably, extra-peritoneal) surgical corridor is developed through a plane between the ipsilateral psoas major muscle and at least a segment of the ipsilateral quadratus lumborum muscle. The ipsilateral transverse process of one or both vertebral bones of the target functional spinal unit is removed. If desired, the removed transverse process may be used as a bone graft (i.e., autograft) material to fuse two or more skeletal bones of the individual during the same surgical procedure. Preferably, the harvested transverse process bone is incorporated into the bone graft that is used to fuse the superior vertebral bone to the inferior vertebral bone of said target functional spinal unit. That is, at least a portion of the bone graft that is used to fuse the superior to the inferior vertebral bones (by positioning a segment of the bone graft to abut the superior vertebrate bone and a segment to abut the inferior vertebral bone) is comprised of bone derived from the harvested transverse process.

At least a portion of the harvested transverse process bone may be preferably, but not necessarily, placed into the target intervertebral disc space in order to form an interbody fusion within the target functional spinal unit. Further, bone graft material (whether containing autograft bone, allograft bone, a synthetic material, or any other substance adapted to form bone) may be placed to extend along the longitudinal axis of the spine from the lateral aspect of the superior articular process (SAP) of the superior vertebral bone to the superior articular process (SAP) of the inferior vertebral bones of the target functional spinal unit. The bone graft material will eventually form a fusion mass that connects the SAP and transverse processes (or the remaining stump thereof) of adjacent vertebral bones (FIG. 26B).

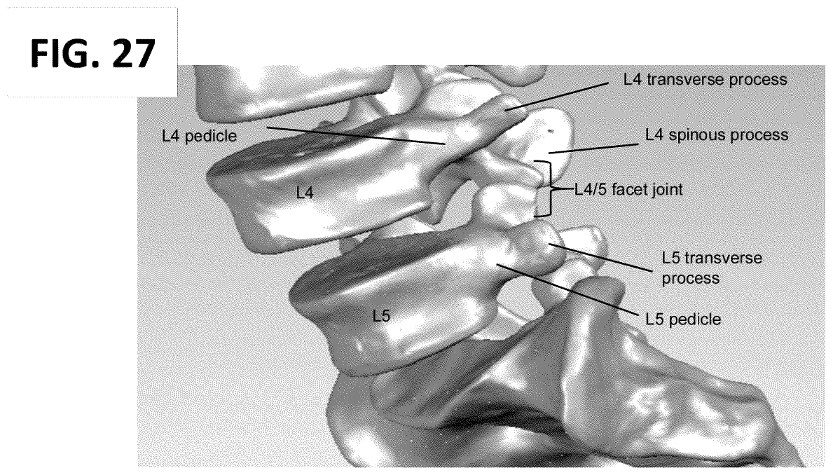

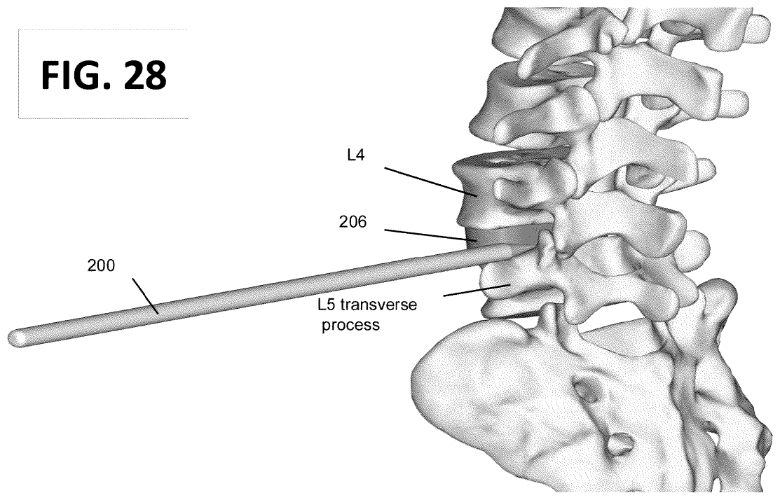

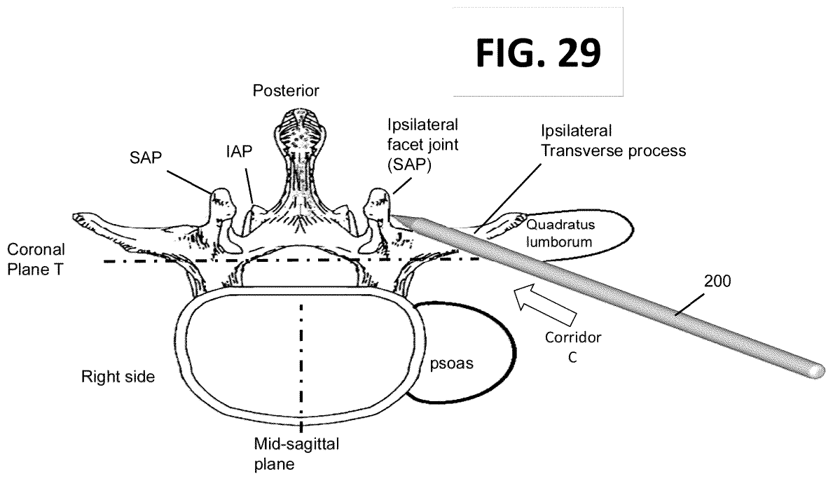

A facet joint, by definition, is an articulation comprised of the superior articulating process (SAP) of an inferior vertebral bone and the inferior articulating process (IAP) of the immediately superior vertebral bone. In the target functional spinal unit, a right and a left facet joints form articulations between the superior and inferior vertebral bone—with a single facet joint on each side of the mid sagittal plane of the vertebral column (FIG. 27). Using corridor C to reach the ipsilateral transverse process, as described above, the ipsilateral facet joint (ipsilateral to said skin incision) can be also accessed. Preferably, but not necessarily, the ipsilateral transverse process of the inferior vertebral bone of the target functional spinal unit is removed in order to provide a wider corridor through which to access said ipsilateral facet joint. However, it is understood that the transverse process may be left in place or only partially removed and the ipsilateral facet joint accessed around it. When the transverse process is not fully removed, the facet joint is preferably accessed through an anterior to posterior trajectory that passes superior to said ipsilateral transverse process of the inferior vertebral bone—as shown in FIGS. 28 and 29. The trajectory used to access the ipsilateral facet joint via corridor C will necessarily cross coronal plane T in an anterior to posterior trajectory (FIG. 29) and will substantially follow member 200. Note that the tip of member 200 is positioned at the lateral surface of the SAP of the inferior vertebral bone of the target FSU.

The preceding steps constitute a method to access the ipsilateral facet joint between the superior and inferior vertebral bones of a target functional spinal unit. Once accessed, the ipsilateral facet joint may be at least partially removed, if desired, to decompress the nerve elements. The joint, whether whole or after partial resection, may be also implanted with fastener(s) that serve to limit and/or completely immobilize movement between the said superior and inferior vertebral bones, as will be further illustrated below.

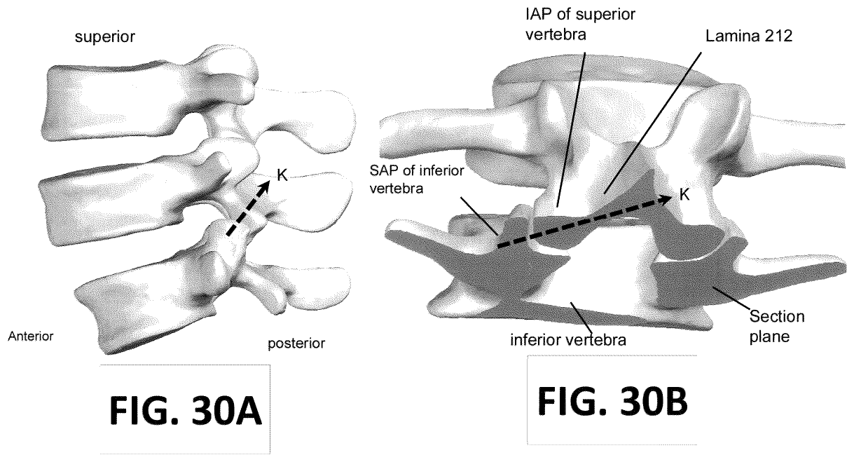

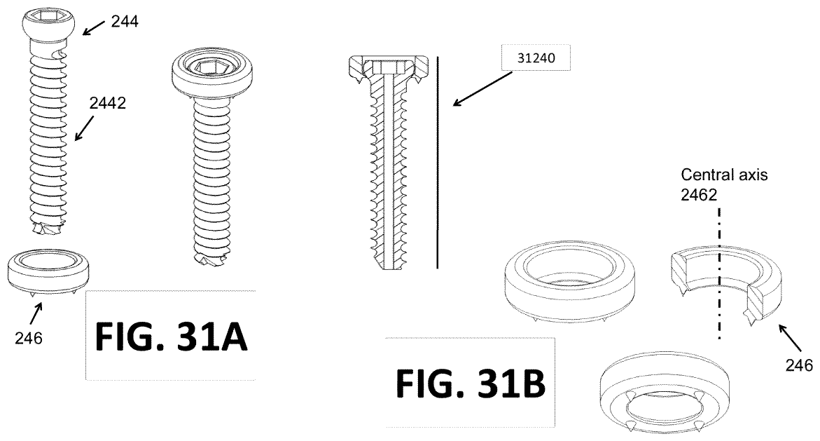

After the ipsilateral facet is accessed through corridor C, a fastener may be placed into the ipsilateral facet joint in order to immobilize the movement between the superior and inferior vertebral bones across said joint. Following a lateral to medial trajectory (such as, for example, the trajectory of member 200), the fastener may be passed through the lateral aspect of the SAP of the inferior vertebral bone, across the facet joint space and into the IAP of the superior vertebral bone—as shown by arrow K in FIGS. 30A and 30B. Note that the fastener may be further passed into the ipsilateral lamina 212 of the superior vertebra as shown in FIG. 30B. FIG. 30B illustrates sectional view passing through the facet joints between the L3 and L4 vertebral bones. The plane of the sectional view is shown by the anterior-posterior direction of arrow K of FIG. 30A. The lateral-medial direction of arrow is shown in FIG. 30B.

The disclosed device embodiments or any of their components can be made of any biologically adaptable or compatible materials. Materials considered acceptable for biological implantation are well known and include, but are not limited to, stainless steel, titanium, tantalum, combination metallic alloys, various plastics (such as PEEK and the like), resins, ceramics, biologically absorbable materials and the like. Any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, any surface may be made with a porous ingrowth surface (such as, for example, porous titanium, titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. The system or any of its components may be made by “additive manufacturing”, such as, for example, “3D” printing. Lastly, the system or any of its components can also be entirely or partially made of a shape memory material or other deformable material.

### Methods

One embodiment of a method of use for the implant assemblies disclosed herein includes inserting an implant assembly into the target intervertebral disc space using any desired surgical approach to the spine (such as those described supra). The assembly comprises a first (host) member and a second member comprising a distraction mechanism. The first member comprises at least a first and a second segment that are configured to move relative to one another, wherein, for example, movement of the first segment relative to the second segment apart increases a height, a length, and/or a width of the first member. Preferably, but not necessarily, the first member does not comprise its own distraction mechanism; however in some examples, the first member may comprise an integral distraction mechanism. In one variant, the method further includes anchoring one of upper and lower elements of the first member to one of the superior and inferior vertebral bones of the target FSU (such as, for example, by via a first bone screw). Further, the method may include anchoring the other of the upper and lower elements of the first member to the other of the superior and inferior vertebral bones of the target FSU (such as, for example, by via a second bone screw).

The method further includes actuating the distraction mechanism after positioning of the implant assembly at the desired location within the target disc space. The first member and the second member are coupled such the act of actuating of the distraction mechanism (the second member) causes a distance between the upper and lower elements to increase on at least one end or side of the first member. Additionally, a distance between one or more portions of the upper and lower elements may decrease, and/or both ends a distance between the upper and lower elements at both ends of the first member may increase. For example, the upper and lower elements the first member to move away from (or towards) one another. In another example, one of the upper and lower elements may move away from (or towards) the other of the upper and lower elements. In this way, the act of actuating of the distraction mechanism of the second member increases at least one of the height, length, and/or width dimensions of the implant assembly and alters the distance between the superior and inferior bones within at least one plane (coronal, axial, and/or sagittal planes) of the spine.

### Alternate Configurations

It is appreciated that while the foregoing description describes an implant device 104 that may have one side (but not the other) increased in height, the present disclosure contemplates alternate implant configurations which have more degrees of freedom. For instance, in one such configuration, both sides of the implant can be adjusted as to height, whether increased or decreased, so as to obtain an optimal relative height/size profile for the target space. In one such implementation (not shown), rather than being hinged as shown in FIGS. 6A and 6B, the pivot or axes of rotation of the implant upper and lower segments is disposed more centrally to the implant 104, such that the two sides of the implant can “toggle” or alternate.

In yet another implementation, both sides of the implant can increase or decrease in tandem with one another, such as via use of a hinge or pivot that is centrally located on the implant, yet which can also translate in a direction normal to the plane of the implant device (i.e., such that the upper and lower elements 1051a, 1051b can move closer or further apart from one another, while also having different heights if desired.

In such configurations, more than one distraction member 205 may also be utilized, such as where one distraction member is disposed at or near each end (side) of the implant device.

Once the desired configuration is achieved, the translation mechanism (e.g., a set screw or other mechanism) can be fastened and locked so as to make the configuration effectively permanent (at least during the lifetime of the implantation). Adhesives or yet other means for maintaining the desired position can be utilized as well. In one variant, the distraction mechanism can be removed after fastening or locking of the first member of the desired position.

In another embodiment for the variable height implant, the implantable device comprises at least two members, the first being a body (which may comprise more than one segment) configured to abut each of the superior and inferior vertebral bones of the target FSU containing the target intervertebral disc space. In one implementation, the first member is comprised of a solid material, which may further be malleable so as to facilitate conformance with at least portions of the target space, and is adapted for implantation into the subject. In another implementation, the first member may be at least partially comprised of a balloon or variable-geometry inflatable bladder. The second member comprises a distraction mechanism that provides the force needed to increase the height of one or both lateral sides of the implant, as well as the structure to maintain the increased height of the implant. In one variant, the distraction mechanism comprises a separate member that can be reversibly inserted/coupled onto the first member by the operator at the time of the surgical procedure.

In another variant, the second member is permanently attached to or integrally formed with the first member. In one implementation, the mechanism comprises an actuatable mechanical device, such as a hydraulic piston. In another implementation, the mechanism is at least partially comprised of a balloon.

In still another embodiment, the implant device is a kit comprised of at least the first and second members. In one implementation, the first and second members can be used alone or in combination depending on the specific condition or configuration of a spine of a patient.

The disclosed device embodiments or any of their components can be made of any biologically adaptable or compatible materials. Materials considered acceptable for biological implantation are well known and include, but are not limited to, stainless steel, titanium, tantalum, combination metallic alloys, various plastics (such as PEEK and the like), resins, ceramics, biologically absorbable materials and the like.

Any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation.

Further, any surface may be made with a porous ingrowth surface (such as, for example, porous titanium, titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. The system or any of its components may be made by “additive manufacturing”, such as, for example, “3D” printing.

Lastly, the system or any of its components can also be entirely or partially made of a shape memory material or other deformable material.

While this specification contains certain specific features and attributes, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

It will also be recognized that while certain aspects of the disclosure are described in terms of a specific sequence of steps of a method, these descriptions are only illustrative of the broader methods of the disclosure, and may be modified as required by the particular application. Certain steps may be rendered unnecessary or optional under certain circumstances. Additionally, certain steps or functionality may be added to the disclosed embodiments, or the order of performance of two or more steps permuted. All such variations are considered to be encompassed within the disclosure disclosed and claimed herein.

1.-26. (canceled)

27. A device assembly for treatment of a spinal segment of a subject, the spinal segment extending along a longitudinal axis of the subject and comprising a superior vertebral bone, an inferior vertebral bone, and an intervertebral disc space positioned there between, the device assembly comprising:
a first member comprising at least a top element configured to a engage a lower surface of the superior vertebral bone, an opposing bottom element, a first member configured to interconnect the top element and the bottom element, and a first cavity at least partially defined by the first member; and
a second member comprising: (i) a top surface configured to, when the second member is at least partially received within the cavity, engage the top element of the first member; and (ii) a bottom surface configured to when the second member is at least partially received within the cavity, engage the bottom element of the first member; and
a distraction apparatus configured to, upon actuation, controllably vary a first distance between the top surface and the bottom surface, the variation of the distance further causing variation of a second distance between the top element and the bottom element of the first member to a desired value.

28. The device assembly of claim 27, wherein the top element and bottom element are hinged or cantilevered such that the actuation of the distraction apparatus causes a first end of the first member to vary in height by a different amount than a second end of the first member varies.

29. The device assembly of claim 28, wherein the cavity is disposed more towards the first end of the first member than the second end.

30. The device assembly of claim 27, wherein the cavity is oriented such that the second member can be inserted at least partially with the cavity when the first member is disposed within the intervertebral disc space, the insertion via a side surface of the intervertebral disc space.

31. The device assembly of claim 27, wherein the top element and bottom element are hinged or cantilevered relative to one another, and the first member configured to interconnect the top element and the bottom element comprises a member configured to rotatably interconnect the top element and bottom element via a first end of the first member.

32. A method for use of an implantable device to treat an intervertebral disc space of a spinal segment of a subject, the spinal segment extending along a longitudinal axis of the subject and comprising a superior vertebral bone, an inferior vertebral bone and the intervertebral disc space positioned there between, the method comprising:
advancing a first member of the implantable device at least partially into the intervertebral disc space, the first member comprising (i) an outer perimeter, the outer perimeter comprising a first top surface configured to a abut a lower surface of the superior vertebral bone, (ii) a first bottom surface positioned opposite the first top surface, (iii) a first vertical member configured to interconnect and separate the first top surface and the first bottom surface, and (iv) a first cavity at least partially formed within the first member; and
selecting a second member of the implantable device, the second member comprising an assembly sized to be at least partially received within the first cavity of the first member, the second member further comprising a second top surface, an opposing second bottom surface, and a distraction mechanism configured to, upon actuation, vary a distance between the second top surface and the second bottom surface;
positioning a bone forming material at least partially within the first member; and
wherein the act of advancing the second member into the first cavity produces an increase in a value of at least one external dimension of the outer perimeter of first member via at least engagement of the second top surface and the second bottom surface with the first member.
